(19) United States
(12) Patent Application Publication
(10) Pub. No.: US 2021/0371862 A1
(43) Pub. Date: Dec. 2, 2021

(54) METHOD AND MEANS TO DELIVER miRNA TO TARGET CELLS

(71) Applicant: Uniqure IP B.V., Amsterdam (NL)

(72) Inventors: Astrid VALLES-SANCHEZ, Amsterdam (NL); Pavlina Stefanova KONSTANTINOVA, Amsterdam (NL); Sander Jan Hendrik VAN DEVENTER, Amsterdam (NL); Marina Sogorb GONZÁLEZ, Amsterdam (NL)

(21) Appl. No.: 17/317,688

(22) Filed: May 11, 2021

Related U.S. Application Data
(63) Continuation of application No. PCT/EP2019/081822, filed on Nov. 19, 2019.
(60) Provisional application No. 62/769,111, filed on Nov. 19, 2018.

(30) Foreign Application Priority Data
Nov. 19, 2018 (EP) 18206970.8

(51) Int. Cl. C12N 15/113; C12N 15/86; A61P 25/28; A61K 9/00; A61K 31/713

(52) U.S. Cl. CPC C12N 15/113; C12N 15/86; A61P 25/28; A61K 9/0085; C12N 2750/14171; C12N 2310/141; C12N 2320/32; C12N 2750/14143; A61K 31/713

(57) ABSTRACT

The invention relates to the field of gene therapy. In addition the invention relates to the field of interfering RNA and/or microRNA (miRNA). In particular the invention relates to gene therapy involving such miRNA's and more in particular to methods and means to improve delivery of said miRNAs to target cells of a patient. The invention provides for a gene delivery vehicle for use in delivery of a miRNA to a cell resulting in silencing of a desired gene and whereby spread of said miRNA to other non-transduced cells results in silencing of said desired gene in said non-transduced cells.

### CROSS-REFERENCE TO RELATED APPLICATIONS

This application is a continuation of International Application No. PCT/EP2019/081822, filed Nov. 19, 2019, which claims the benefit of and priority to European Application No. 18206970.8, filed Nov. 19, 2018, and U.S. Provisional Patent Application No. 62/769,111 filed Nov. 19, 2018, all of which are hereby incorporated by reference herein in their entireties.

### FIELD OF THE INVENTION

The invention relates to the field of gene therapy. In addition the invention relates to the field of interfering RNA and/or microRNA (miRNA). In particular the invention relates to gene therapy involving such miRNA's and more in particular to methods and means to improve delivery of said miRNAs to target cells of a patient. More in particular the invention relates to the treatment of diseases including neurodegenerative diseases such as Huntington or spinocerebellar ataxia type 3 (SCA3).

### SEQUENCE LISTING

The instant application contains a Sequence Listing which has been submitted-electronically in ASCII format and is hereby incorporated by reference in its entirety. Said ASCII copy, created on Aug. 20, 2021, is named 069818-0630_SL.txt and is 11,742 bytes in size.

### BACKGROUND

Gene Therapy

The elucidation of DNA as the carrier of genetic information, and therefore also as the source of inherited diseases, has led to envisaged therapies in which mutant, damaged genes could be replaced or at least silenced. Many genes and/or other nucleic acid sequences have now been identified to play a role in (genetic) disease. If the mutant gene(s) could be replaced by a healthy one, or if the genes expressing aberrant (sometimes toxic) products could be silenced the disease could be treated at the molecular level, and, potentially be cured. Gene therapy provides a promising concept, in particular for diseases caused by mutations in a single gene.

However, the delivery of the desired nucleic acid to the cells that need to be targeted is not an easy task. Numerous (viral) delivery systems have been investigated, all of them with their own advantages and drawbacks. One of the viral delivery vehicles that is used for gene therapy is Adeno Associated Virus.

AAV

AAV has a single-stranded DNA genome of approximately 4.8 kilobases (kb). AAV belongs to the parvovirus family and is dependent for replication on co-infection with other viruses, in particular adenoviruses. The genome comprises, Rep (Replication) and Cap (Capsid) genes. These coding sequences are flanked by inverted terminal repeats (ITRs) that are required for genome replication and packaging. The Rep gene encodes four proteins (Rep78, Rep68, Rep52, and Rep40), replicate the viral genome and facilitate packaging, while Cap expression gives rise to the viral capsid proteins (VP; VP1/VP2/VP3), which form the outer capsid shell.

For gene therapy the viral DNA of the AAV is almost completely removed. Recombinant AAV (rAAV) for gene therapy is formed by a protein capsid containing a desired nucleic acid, the transgene, that is to be delivered to target cells. The desired nucleic acid is flanked with the ITR's of AAV. ITR-flanked transgenes encoded by rAAV can form circular concatemers remaining in the nucleus of transduced cells as episomes. As the episome remains largely episomal the expression of AAV delivered nucleic acid sequences may be diluted over time if and when the target cell replicates. This dilution may not generally apply to post-mitotic cells such as neurons, which are the target cells for many neurodegenerative diseases. A recent review on AAV vectors for gene therapy is provided in Naso et al, Biodrugs 2017 (p. 317-334).

miRNA

RNA interference (RNAi) is a naturally occurring mechanism that involves sequence specific down regulation of messenger RNA (mRNA). The down regulation of mRNA results in a reduction of the amount of protein that is expressed. RNA interference is triggered by double stranded RNA. One of the strands of the double stranded RNA is substantially or completely complementary to its target, the mRNA. This strand is termed the guide strand. The mechanism of RNA interference involves the incorporation of the guide strand in the RNA-induced silencing complex (RISC). This complex is a multiple turnover complex that via complementary base paring of the guide strand can bind to its target mRNA. Once bound to its target mRNA it can either cleave the mRNA or reduce translation efficiency. RNA interference has since its discovery been widely used to knock down specific target genes. The triggers for inducing RNA interference that have been employed involve the use of small interfering RNAs (siRNAs) or short hairpin RNAs (shRNAs). In addition, molecules that can naturally trigger RNAi, the so called microRNAs (miRNAs), have been used to make artificial (engineered) miRNAs that mimic their naturally occurring counterparts. These strategies have in common that they provide for substantially double stranded RNA molecules that are designed to target a mRNA of choice. RNAi based therapeutic approaches that utilise the sequence specific modality of RNAi are under development and several are currently in clinical trials (see i.a. Davidson and McCray, Nature Reviews—Genetics, 2011; Vol. 12; 329-340).

Huntington's Disease

The huntingtin gene, also referred to as the HTT or HD (Huntington's disease) gene, encodes for the huntingtin mRNA and protein. The huntingtin gene is a large gene on chromosome 4p. 13 of about 13.5 kb (huntingtin protein is about 350 kDa). Huntington's disease is a genetic neurodegenerative disorder caused by a genetic mutation in the huntingtin gene. The genetic mutation involves a DNA segment of the huntingtin gene known as the CAG trinucleotide repeat. Normally, the CAG segment in the huntingtin gene of humans is repeated multiple times, i.e. about 10-35 times. People that develop Huntington's disease have an expansion of the number of CAG repeats in at least one allele. An affected person usually inherits the mutated allele from one affected parent. In rare cases, an individual with Huntington's disease does not have a parent with the disorder (sporadic HD). People with 36 to 39 CAG repeats may develop signs and symptoms of HD, while people with 40 or more repeats always develop the disorder, marked by a triad of motor, cognitive and psychiatric symptoms that ultimately leads to death. The increase in the size of the CAG repeat leads to the production of an aberrant HTT mRNA resulting in a RNA toxic gain-of-function, and to a production of mutant huntingtin protein with an elongated polyglutamine (polyQ) stretch. The mutant huntingtin protein is processed in the cell into smaller fragments that are cytotoxic and that accumulate and aggregate in neurons, starting in the striatum and in the cerebral cortex in later stages of the disease. This results in the disruption of normal function and eventual death of neurons. This is the main process that occurs in the brain which underlies the signs and symptoms of Huntington's disease.

SCA3

Spinocerebellar ataxia type 3 (SCA3), or Machado-Joseph disease (MJD), is an autosomal dominant monogenic, fatal disorder. The disorder is characterized by progressive degeneration of brain areas, which is caused by a CAG expansion in the human ataxin-3 gene, also referred to as ATXN3 gene (OMIM: 607047, reference sequence Homo sapiens ataxin 3 (ATXN3) on chromosome 14, NCBI Reference Sequence: NG_008198.2. In the 3′ region of the gene, a cytosine-adenine-guanine (CAG) repeat region is present. Said CAG region is in frame and results in an ataxin-3 protein comprising a polyQ region, a repetitive sequence of glutamines. Healthy, or non-symptomatic, individuals may have up to 44 CAG-repeats in the ATXN3 gene. Diseased individuals have expansions and it has been shown that they may have between 52 and 86 or more CAG repeats. Individuals having between 45-51 CAG repeats are to have symptoms with incomplete penetrance of disease. Said expansion resulting in ataxin-3 protein that have extended polyQ regions and the length of the CAG repeats, and thus polyQ regions within ataxin-3, can be correlated with disease progression, i.e. the longer the region usually the more progressive the disease.

The ataxin-3 protein with the expanded polyQ tract acquires toxic properties (gain of toxic function) and the formation of neuronal aggregates in the brain is the neuropathological hallmark. Neuropathological studies have detected widespread neuronal loss in various areas, including cerebellum, thalamus, midbrain, pons, medulla oblongata and spinal cord of SCA3 patients (Riess et al., Cerebellum 2008). Although widespread pathology is reported, the consensus is that the main pathology is in the cerebellum and in the brainstem (Eichler et al. AJNR Am J Neuroradiol, 2011). The disease has full penetration, which means that if a person has an expansion of 52 or more CAGs, they will inevitably develop the disease and have 50% chance to pass it on to their offspring.

### SUMMARY OF THE INVENTION

According to the invention methods and means are provided that deliver a source of miRNA to a target cell, but that, in addition, lead to release of said miRNA from said target cell by incorporation of said miRNA in extracellular vesicles, in particular exosomes and microvesicles. Hence, said miRNA being comprised in an extracellular vesicle may subsequently be delivered to cells that are not necessarily provided with the source of the miRNA. Hence, the induced silencing by said miRNA in said target cell is not restricted to the target cells but spreads beyond said target cells. Thus, according to the invention methods and means are provided that deliver a source of miRNA to a target cell, but that, in addition, lead to spread of said miRNA from said target cell to other cells by incorporation of said miRNA in extracellular vesicles, in particular exosomes and microvesicles. The methods and means of the invention are useful for the treatment of disease, preferably in humans. The methods and means of the invention are particularly useful for the treatment of neurodegenerative diseases, in particular Huntington's disease, amyotrophic lateral sclerosis (ALS), spinocerebellar ataxia, Parkinson's disease and Alzheimer's disease.

### EMBODIMENTS

Over the last couple of years it has been shown that extracellular vesicles, and in particular exosomes and microvesicles, do not just have a function in waste disposal, but play a role in transporting molecules from one cell to another and thereby have possible roles in signalling and regulation. As shown in the examples herein, miRNAs originating from a transgene present in a cell are also incorporated into extracellular vesicles. Such extracellular vesicles containing miRNAs (possibly in a complex, such as in RISC) can fuse with the membrane of other cells and deliver the miRNA to said cell. The miRNA can silence its target in the cell with which the extracellular vesicle has fused. Also shown herein is that the level of silencing that is seen in primate subjects transduced with gene delivery vehicles containing certain scaffolds for carrying miRNA is higher than expected based on the amount of gene delivery vehicle provided to the subject. This indicates that the spreading that is seen in vitro may also occur in vivo.

The present inventors have established that certain miRNA scaffolds used in gene therapy have a greater tendency to be packaged into extracellular vesicles than other miRNA scaffolds. The present inventors observe that certain miRNA scaffolds used in gene therapy may have a greater tendency to be packaged into extracellular vesicles than other miRNA scaffolds and that certain miRNA sequences may be packaged into extracellular vesicles at a higher rate than others.

It is therefore an object of the present invention to provide a gene delivery vehicle for use in delivery of a miRNA to a cell resulting in silencing of a desired gene and whereby spread of said miRNA to other non-transduced cells results in silencing of said desired gene in said non-transduced cells. According to the invention said gene delivery vehicle will be preferably for use in primates, such as humans.

According to the invention an artificial miRNA is defined as a sequence that results from replacing a miRNA sequence found in nature with another sequence as it is comprised in its precursor, i.e. a pre-miRNA or a pri-miRNA sequence. The miRNA produced from said scaffold no longer being the miRNA as found in nature (not the original sequence, hence the term artificial). MicroRNAs, i.e. miRNA, are guide strands that originate from double stranded RNA molecules that are endogenously expressed e.g. in mammalian cells. A miRNA is processed from a pre-miRNA precursor molecule, by the RNAi machinery and incorporated in an activated RNA-induced silencing complex (RISC) (Tijsterman M, Plasterk R H. Dicers at RISC; the mechanism of RNAi. Cell. 2004 Apr. 2; 1 17(1):1-3). A pre-miRNA is a hairpin RNA molecule that can be part of a larger RNA molecule (pri-miRNA), e.g. comprised in an intron, which is first processed by Drosha to form a pre-miRNA hairpin molecule. The pre-miRNA molecule is an shRNA-like molecule that can subsequently be processed by Dicer to result in an siRNA-like double stranded RNA duplex. One endogenous miRNA, miR451 does not require Dicer for processing, but it is instead processed by the Argonaute 2 (Ago2) enzyme and subsequently trimmed by the Poly(A)-specific ribonuclease (PARN) to the mature 22/26-nt miR451 (Herrera-Carrillo and Berkhout, Nucleic Acids Res, 2017, 45(18):10369-10379). The resulting mature miRNA, obtained either by Dicer-dependent or by Dicer-independent processing, i.e. the guide strand, that is part of the double stranded RNA duplex is subsequently incorporated in RISC. An RNA molecule such as present in nature, i.e. a pri-miRNA, a pre-miRNA or a miRNA duplex, may be used as a scaffold for producing an artificial miRNA that specifically targets a gene of choice. Based on the predicted RNA structure of the RNA molecule as present in nature, e.g. as predicted using e.g. m-fold software using standard settings (Zuker. Nucleic Acids Res. 31 (13), 3406-3415, 2003), the natural miRNA sequence as it is present in the RNA structure (i.e. duplex, pre-miRNA or pri-miRNA), and the sequence present in the structure that is substantially complementary therewith are removed and replaced with a first RNA sequence and a second RNA sequence. The first RNA sequence and the second RNA sequence are preferably selected such that the predicted secondary RNA structures that are formed, i.e. of the pre-miRNA, pri-miRNA and/or miRNA duplex, resemble the corresponding predicted original secondary structure of the natural RNA sequences. pre-miRNA, pri-miRNA and miRNA duplexes (that consist of two RNA strands that are hybridized via complementary base pairing) as found in nature often are not fully base paired, i.e. not all nucleotides that correspond with the first and second strand as defined above are base paired, and the first and second strand are often not of the same length. How to use miRNA precursor molecules as scaffolds for any selected target RNA sequence and substantially complementary first RNA sequence is described e.g. in Liu Y P Nucleic Acids Res. 2008 May; 36(9):281 1-24, which is incorporated herein by reference.

Whatever design is used for the miRNA scaffold, which is preferably based on miR451, it is designed such that therefrom an antisense RNA molecule comprising the first RNA sequence, i.e. the sequence that replaced the original miRNA sequence, in whole or a substantial part thereof, can be processed by the RNAi machinery such that it is incorporated in the RISC complex to have its action, i.e. to induce RNAi e.g. against the RNA target sequence comprised in an RNA encoded by gene associated with a disease. The artificial miRNA that is produced from the miRNA scaffold is thus not necessarily identical in sequence length to the sequence that is used to replace the endogenous miRNA sequence. The artificial miRNA that is produced from the miRNA scaffold also not necessarily comprises the exact sequence that is used to replace the wild-type miRNA sequence. Thus, the miRNA sequence comprises or consists of the first RNA sequence, or the miRNA sequence comprises in whole or a substantial part of the first RNA sequence, said miRNA sequence being capable of sequence specifically targeting a gene, e.g. a gene transcript. Hence, as long as the miRNA produced from the miRNA scaffold is capable of inducing RNAi, such a scaffold is part of the invention. The artificial miRNA may thus preferably be comprised in a pre-miRNA scaffold or a pri-miRNA scaffold.

As shown in the examples, an artificial miRNA may be preferably incorporated in a pre-miRNA or a pri-miRNA scaffold derived from microRNA 451a. The terms ‘microRNA451a’, ‘miR451’, ‘451 scaffold’ or simply ‘451’ are used interchangeably throughout this specification. This scaffold allows to induce RNA interference resulting in only guide strand induced RNA interference. The pri-miR451 scaffold does not result in a passenger strand because the processing is different from the canonical miRNA processing pathway (Cheloufi et al., Nature 2010 Jun. 3; 465(7298):584-9; Cifuentes et al, Science, 2010, 328 (5986), 1694-1698 and Yang et al., Proc Natl Acad Sci USA. 2010 Aug. 24; 107(34):15163-8). Hence, this scaffold represents a preferred embodiment of the invention to produce a gene therapy product according to the invention, as unwanted potential off-targeting by passenger strands can be largely, if not completely, avoided.

As an alternative to the miR451 scaffold, similar Dicer independent structures may be preferably employed such as described herein and i.a. in Herrera-Carrillo and Berkhout, Nucleic Acids Res, 2017, Vol. 45 No. 18 10369-79, which is incorporated herein by reference. As a passenger strand may result in off-targeting e.g. targeting transcripts other than the desired target, using such a scaffold may allow one to avoid such unwanted targeting.

As shown in the examples, a first RNA sequence of 22 nucleotides (e.g. for a miR451) in length may be selected and incorporated in a miRNA scaffold. Such a miRNA scaffold sequence is subsequently processed by the RNAi machinery as present in the cell. When reference is made to miRNA scaffold it is understood to comprise pri-miRNA structures or pre-miRNA structures. miRNA scaffolds based on 451, when processed in a neuronal cell, can result in guide sequences, i.e. an artificial miRNA, comprising the first RNA sequence (the sequence that replaced the endogenous 451 miRNA sequence) or a substantial part thereof, having a length which is in the range of 19-30 nucleotides as shown in the examples. Such guide strands are capable of reducing the target gene expression by targeting the selected target sequences. As is clear from the above, the first RNA sequence as it is encoded by the expression cassette of the invention, is comprised in part or in whole, in a guide strand when it has been processed by the RNAi machinery of the cell. Hence, the guide strand, i.e. artificial miRNA, that is to be generated from the RNA encoded by the expression cassette, comprising the first RNA sequence and the second RNA sequence is to comprise at least 18 nucleotides of the first RNA sequence. Preferably, such a guide strand comprises at least 19 nucleotides, 20 nucleotides, 21 nucleotides, or at least 22 nucleotides. A guide strand can comprise the first RNA sequence also as a whole. In selecting a miRNA scaffold e.g. from miRNA scaffolds as found in nature such as in humans, the first RNA sequence can be selected such that it is to replace the original guide strand. This does not necessarily mean that a guide strand produced from such an artificial scaffold are identical in length and sequence to the first RNA sequence selected, nor may it necessarily be so that the first RNA sequence is in its entirety to be found in the guide strand that is produced.

A miRNA 451 scaffold, preferably comprises from 5′ to 3′, firstly 5′-CUUGGGAAUGGCAAGG-3′ (SEQ ID NO. 20), followed by a sequence of 22 nucleotides, comprising or consisting of the first RNA sequence, followed by a sequence of 17 nucleotides, which is complementary over its entire length with nucleotides 2-18 of said sequence of 22 nucleotides, subsequently followed by sequence 5′-CUCUUGCUAUACCCAGA-3′ (SEQ ID NO. 21). Preferably the first 5′-C nucleotide of the latter sequence is not to base pair with the first nucleotide of the first RNA sequence. Such a scaffold may comprise further flanking sequences as found in the original pri-miR451 scaffold. Alternatively, the flanking sequences, may be replaced by flanking sequences of other pri-mRNA structures. It is understood that, as the miR451 scaffold can provide for guide strands only due to the length of the stem sequence, it is preferred that alternative flanking sequences do not extend the stem length of 17 consecutive base pairs. As is clear from the above, the sequence of the scaffold may differ not only with regard to the (putative) guide strand sequence, and sequence complementary thereto, as present in the wild-type scaffold, but may also comprise additional mutations in the 5′sequence, loop sequence and 3′ sequence as well, as additional mutations may be required to provide for an RNA structure that is predicted to mimic the secondary structure of the wild-type scaffold and/or does not have a stem extending beyond 17 consecutive base pairs. Such a scaffold may be comprised in a larger RNA transcript, e.g. a pol II expressed transcript, comprising e.g. a 5′ UTR and a 3′UTR and a poly A. Flanking structures may also be absent. An expression cassette in accordance with the invention may thus express a shRNA-like structure having a sequence of 22 nucleotides, comprising or consisting of the first RNA sequence, followed by a sequence of 17 nucleotides, which is complementary over its entire length with nucleotides 2-18 of said sequence of 22 nucleotides, and further comprising 1 or more additional nucleotides which is predicted not to form a base pair with the first RNA sequence. The latter shRNA-like structure derived from the miR451 scaffold can be referred to as a pre-miRNA scaffold from miR451.

The gene delivery vehicle may be non-viral or viral. It is for instance possible to use extracellular vesicles themselves as the primary gene delivery vehicle, if the extracellular vesicles are loaded with the genetic information for expression of microRNAs on scaffolds that together lead to significant production of extracellular vesicles by transduced cells. Both miRNA and scaffold may contribute to the preferential packaging into extracellular vesicles by transduced cells. As has been done in our in vitro experiments, the skilled person can easily assess whether miRNAs and/or their scaffolds lead to significant packaging in extracellular vesicles. It is known how to transduce cells with a gene delivery vehicle. It is also known how to isolate extracellular vesicles (Li et al, Theranostics, vol 7(3) p. 789-804, 2017). Techniques to determine the abundance of the miRNA (complexed or not) in the isolated extracellular vesicles are generally available. In this way the skilled person can determine which scaffolds, which miRNAs and which combinations thereof are suitable candidates for use in a gene delivery vehicle according to the invention.

In a preferred embodiment a scaffold is used that is based on mir451 that has been shown to lead to high levels of miRNA products in extracellular vesicles.

Thus a preferred embodiment of the invention provides a gene delivery vehicle for use in delivery of an miRNA to a cell resulting in silencing of a desired gene and spread of said miRNA to other cells for silencing of said desired gene, wherein said gene delivery vehicle comprises a nucleic acid encoding a mir451 scaffold or a functional equivalent thereof.

As said, according to the invention the spreading (or bystander effect) occurs through packaging of the miRNA in extracellular vesicles, in particular exosomes or microvesicles. The extracellular vesicles produced by transduced cells will be released into the environment of the transduced cell and may fuse with neighbouring cells, or be transported through the subject to fuse with more distant cells. This mechanism also allows for treatment protocols where the gene delivery vehicle is administered to a set of cells in a certain location of the body of the subject, but can through spreading reach other areas of the body of the subject.

Furthermore, said spreading may also occur through further means. For example, as shown in the example section, miRNAs are not only associated with extracellular vesicles, but are also found in fractions associated with proteins and high-density lipoproteins (HDLP). Hence, accordingly said spreading in accordance with the invention, may also occur with said miRNA being comprised in such protein and/or HDLP fractions.

In one embodiment the gene delivery vehicle according to the invention will result in nuclear silencing. Nuclear silencing means that the silencing takes place in the nucleus of a cell. This has the advantage that e.g. unspliced messengers can be targeted, and by avoiding the generation of mutated RNAs in certain diseases, generation of RNA toxic species could significantly be reduced. Diseases in which e.g. unspliced messengers are associated with a disease could benefit from such silencing, and in which RNA toxic species are formed. For example, single-nucleotide polymorphisms (SNPs) in cis with the huntington gene mutation largely annotate Huntington's disease haplotypes (Kay et al., Clin Genet. 2014 July; 86(1):29-36). These heterozygous SNPs allow for a design of miRNAs to induce allele-specific lowering. Some of these SNPs are located in the intronic region, resulting in most of the corresponding transcripts being located in the nucleus, and hence it would be advantageous to target these unspliced messenger RNAs in the nucleus. Also in Huntington's disease, RNA toxic species have been suggested to play a role in the neurodegeneration observed in the course of the disease (Marti E et al., Brain Pathol. 2016 November; 26(6):779-786), and this has been observed in other neurodegenerative disorders such as amyotrophic lateral sclerosis (ALS) and frontotemporal dementia (FTD) (Donnelly et al., Neuron. 2013 Oct. 16; 80(2):415-28). In Huntington's disease, RNA pathogenic mechanisms include perturbation of alternative splicing, altered gene silencing, aberrant subcellular localization of transcripts, and nucleolar stress. Some of the RNA toxic species are thus found in the nucleus. Therefore, the invention provides a gene delivery vehicle for use according to the invention wherein said silencing comprises nuclear silencing.

In one embodiment the gene delivery vehicle according to the invention will result in cytoplasmic silencing. This has the advantage that generally the most abundant messenger RNA species are present in the cytoplasm, and therefore they will efficiently be recognized by the artificial miRNA, with efficient engagement of the RNAi machinery leading to silencing of said messenger RNA. Therefore the invention provides a gene delivery vehicle for use according to the invention wherein said silencing comprises cytoplasmic silencing.

According to the invention the gene delivery vehicle may be provided with molecules associated with the surface of the gene delivery vehicle that specifically recognize a molecule associated with the surface of a particular target cell.

As stated herein before there are many known gene delivery vehicles, both viral and non-viral. Said gene delivery vector is to comprise an expression cassette comprising the nucleic acid encoding the miRNA in accordance with the invention. Preferably, gene delivery vehicles are used that can stably transfer the nucleic acid and/or expression cassette to cells in a human patient such that expression of the artificial miRNA can be achieved. Suitable vectors may be lentiviral vectors, retrotransposon based vector systems, or AAV vectors. It is understood that as e.g. lentiviral vectors carry an RNA genome, the RNA genome (a nucleic acid) will encode for the said expression cassette such that after transduction of a cell and reverse transcription a double stranded DNA sequence is formed comprising the nucleic acid sequence and/or said expression cassette in accordance with the invention.

All such vehicles are within the scope of the present invention, but the preferred vehicle is based on AAV vectors. AAV sequences that may be used in the present invention for the production of AAV vectors, e.g. as produced in insect or mammalian cell lines, can be derived from the genome of any AAV serotype. The production of AAV vectors comprising an expression cassette of interest is described i.a. in; WO2007/046703, WO2007/148971, WO2009/014445, WO2009/104964, WO2011/122950, WO2013/036118, which are incorporated herein in its entirety. Generally, the AAV serotypes have genomic sequences of significant homology at the amino acid and the nucleic acid levels, provide an identical set of genetic functions, produce virions, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). AAV serotypes 1, 2, 3, 4 and 5 may be preferred source of AAV nucleotide sequences for use in the context of the present invention. Preferably the AAV ITR sequences for use in the context of the present invention are derived from AAV1, AAV2, and/or AAV5. Likewise, the Rep52, Rep40, Rep78 and/or Rep68 coding sequences are preferably derived from AAV1, AAV2 and AAV5. The sequences coding for the VP1, VP2, and/or VP3 capsid proteins for use in the context of the present invention may preferably be taken from AAV1, AAV2, AAV3, AAV4, AAV5, AAV7, AAV5, AAV5, AAVrh10 and AAV10 as these are serotypes that are suitable for use in gene therapy, such as for the treatment of the CNS. Also, newly developed AAV-like particles obtained by e.g. capsid shuffling techniques and AAV capsid libraries comprising mutations (insertions, deletions, substitutions), derived from AAV capsid sequences, and selected from such libraries as being suitable for specific target tissue transduction may be contemplated. AAV capsids may consist of VP1, VP2 and VP3 capsid proteins, but may also consist of VP1 and VP3 capsid proteins. AAV capsids may not contain any substantial amount of VP2 capsid protein. This is because the VP2 capsid protein may not be essential for efficient transduction.

A preferred AAV vector that may be used in accordance with the inventions is an AAV vector of serotype 5. AAV of serotype 5 (also referred to as AAV5) has been shown useful for many tissue types and has been shown to be in particularly useful for transducing human neuronal cells. AAV vectors comprising AAV5 capsids can comprise AAV5 VP1, VP2 and VP3 capsid proteins. AAV vectors comprising AAV5 capsids can also comprise AAV5 VP1 and VP3 capsid proteins, while not comprising AAV5 VP2 capsid proteins or at least not comprising any substantial amount of VP2 capsid proteins. In a wild-type derived AAV5 capsid protein, the VP1, VP2 and VP3 capsid proteins comprise identical amino acid sequences at their C-termini. The VP3 sequence is comprised in the VP2 sequence, and the VP2 sequence is comprised in the VP1 sequence. The N-terminal part of the VP1 amino acid sequence that is not contained in the VP2 and VP3 capsid proteins is positioned at the interior of the virion. This N-terminal VP1 sequence may e.g. be exchanged with an N-terminal sequence of another serotype, e.g. from serotype 2, whereas the VP2 and VP3 amino acid sequences may be entirely based on the AAV5 serotype. Such non-natural capsids comprising hybrid VP1 sequences, and such hybrid vectors are also understood to be AAV5 viral vectors in accordance with the invention. Such a hybrid vector of the AAV5 serotype is i.a. described by Urabe et al., J Virol. 2006. Furthermore, AAV5 capsid sequences may also have one or more amino acids inserted or replaced to enhance manufacturing and/or potency of a vector, such as i.a. described in WO2015137802. Such modified AAV5 capsids are also understood to be also of the AAV5 serotype.

AAV (also referred to as AAV vector) is preferred because it may remain episomal for a long time, thus giving prolonged expression, but having a very low integration frequency into the host genome, with a very low risk of undesired integration at undesired sites. As the preferred gene delivery vehicles are intended to treat diseases of the brain, the invention has as a preferred embodiment a method wherein said miRNA expressed in the brain is expressed through the introduction of a gene delivery vehicle in the brain. A preferred route of administration of AAV may be to the cerebrospinal fluid (CSF), i.e. intrathecally, such as described e.g. in WO2015060722 or Watson, et al., Gene Therapy, 2006. An alternative route of administration may be intraparenchymal or subpial administration. The artificial miRNA to be delivered according to the invention is preferably comprised in a 451 scaffold. The miRNA 451 scaffold has been disclosed in WO2011133889 and WO2016102664. It has as one of its advantages that is does not generate passenger strand, but more importantly, the present inventors have shown that it can be used as a scaffold to generate artificial miRNAs that are incorporated into extracellular vesicles quite efficiently, thereby making their use in the present invention preferred.

Therefore the invention provides a gene delivery vehicle for use according to the invention, wherein said gene delivery vehicle is a virus derived particle, most preferably wherein said gene delivery vehicle is an AAV based particle. AAV (adeno associated virus) has a set of features that make it particularly suitable for gene therapy (see Naso et al), including the long time maintenance of expression in target cells without viral material integrating in the host cell genome (possibly in harmful places).

There are a number of targets that can be addressed by the gene delivery vehicle products of the invention. In principle any target that can be beneficially downregulated using interfering RNA, in particular miRNA, is a suitable target according to the invention. It may be preferred to downregulate the expression of a protein that causes health issues, e.g. because it is an aberrant protein, possibly even toxic and/or may form aggregates. It may be also be preferred to downregulate the expression of a transcript that causes health issues, e.g. because it is an aberrant transcript, possibly even toxic and/or may form aggregates.

The properties disclosed above make AAV particularly suitable for treatment of the central nervous system (CNS), such as diseases of the brain and/or of the spinal cord. Chronic CNS diseases, such as neurodegenerative diseases may be in particular contemplated.

Therefore the invention provides in one embodiment a gene delivery vehicle for use according to the invention for the treatment of neurodegenerative diseases. Particularly preferred are gene delivery vehicles for use according to the invention for the treatment of Huntington disease, ALS, spinocerebellar ataxias (SCAs), Parkinson's disease, Alzheimer's disease. In these diseases the miRNA needs to bind to the mRNAs of huntingtin, ataxins, c9orf72 or superoxide dysmutase 1 (SOD1), alpha-synuclein, and tau, respectively per each of the earlier mentioned neurodegenerative disorders, to reduce the production of each of the corresponding proteins.

In yet a further embodiment the invention provides a gene delivery vehicle for use in delivery of a miRNA preferentially to a brain cell resulting in silencing of a desired gene and spread of said miRNA to other cells of the central nervous system for silencing of said desired gene, wherein said gene delivery vehicle comprises a mir-451 scaffold or a functional equivalent thereof. In another further embodiment the invention provides a gene delivery vehicle for use in delivery of a miRNA preferentially to a spinal cord cell resulting in silencing of a desired gene and spread of said miRNA to other cells of the central nervous system for silencing of said desired gene, wherein said gene delivery vehicle comprises a mir-451 scaffold or a functional equivalent thereof.

In a further embodiment the invention provides a gene delivery vehicle for use in delivery of a miRNA preferentially to a brain cell resulting in silencing of a desired gene and spread of said miRNA to other cells of the brain for silencing of said desired gene, wherein said gene delivery vehicle comprises a mir-451 scaffold or a functional equivalent thereof. In a further embodiment the invention provides a gene delivery vehicle for use in delivery of a miRNA preferentially to a spinal cord cell resulting in silencing of a desired gene and spread of said miRNA to other cells of the spinal cord for silencing of said desired gene, wherein said gene delivery vehicle comprises a mir-451 scaffold or a functional equivalent thereof.

In another embodiment, said spread of said miRNA to other cells of the brain includes cells of the brainstem, hippocampus, cerebellum and/or white matter. In another embodiment, said spread of said miRNA to other cells of the brain includes cells of the cerebellum, pons, and/or white matter. In another embodiment, said spread of said miRNA to other cells of the brain includes cells of the thalamus, nucleus accumbens, and/or subcortical regions. In yet a further embodiment, a gene delivery vehicle is provided for use in delivery of a miRNA to a brain cell of the caudate and/or putamen, wherein said spread to other cells of the brain includes cells of the brainstem, hippocampus, cerebellum and/or white matter. In another further embodiment, a gene delivery vehicle is provided for use in delivery of a miRNA to a brain cell of the caudate and/or putamen, wherein said spread to other cells of the brain includes cells of the cerebellum, pons, and/or white matter. In a further embodiment, a gene delivery vehicle is provided for use in delivery of a miRNA to a brain cell of the caudate and/or putamen, wherein said spread to other cells of the brain includes cells of of the thalamus, nucleus accumbens, and/or subcortical regions.

Since many extracellular vesicles are preferentially taken up by the liver, the current inventions may be in particularly suitable for the treatment of diseases involving the liver, including metabolic diseases. The gene delivery vehicle thus may be provided to target cells that allow delivery of the subsequent extracellular vesicles to liver cells that have not been provided with a gene delivery vehicle. The target cells may be cells other than cells of the liver. Hence, in yet a further embodiment the invention provides a gene delivery vehicle for use in delivery of a miRNA preferentially to liver cells resulting in silencing of a desired gene and spread of said miRNA to other cells of the liver for silencing of said desired gene, wherein said gene delivery vehicle comprises a mir-451 scaffold or a functional equivalent thereof. Hence, gene delivery vehicles for use according to the invention may be preferred for the treatment of liver diseases such as dyslipidemias and metabolic diseases. Suitable candidates for lipid lowering are described in Nordestgaard et al., Nat Rev Cardiol. 2018 May; 15(5):261-272.

In another embodiment the invention provides a gene delivery vehicle for use in delivery of a miRNA preferentially to a heart cell resulting in silencing of a desired gene and spread of said miRNA to other heart cells for silencing of said desired gene, wherein said gene delivery vehicle comprises a mir-451 scaffold or a functional equivalent thereof. Suitable candidates for targeting in the heart are described i.a. in Fechner et al. Methods Mol Biol. 2017.

In another embodiment the invention provides a gene delivery vehicle for use in delivery of an miRNA preferentially to a cell in the eye resulting in silencing of a desired gene and spread of said miRNA to other cells in the eye for silencing of said desired gene, wherein said gene delivery vehicle comprises a mir-451 scaffold or a functional equivalent thereof. Suitable candidate for targeting in the eye are described i.a. in Oner et al., Turk J Ophthalmol. 2017.

In yet another embodiment the invention provides a gene delivery vehicle for use in delivery of a miRNA preferentially to a muscle cell resulting in silencing of a desired gene and spread of said miRNA to other muscle cells for silencing of said desired gene, wherein said gene delivery vehicle comprises a mir-451 scaffold or a functional equivalent thereof miRNA (or more general other small RNAs such as siRNA) is also detected in significant amounts in cells that have not been transduced. The production of miRNA's and their packaging into extracellular vesicles to be taken up by other cells is a natural process that should not be completely highjacked by the gene therapy. It is therefore preferred to provide the nucleic acid encoding the miRNA with a promoter directing the expression of the miRNA product that is not strong enough to provide such high amounts of therapeutic miRNA that the normal processes are inhibited to a level threatening to silence the normal miRNA processes in a subject. The strength of the promoter necessary can be determined ex vivo by determining the relative abundance of the therapeutic miRNA when compared with other miRNA products for instance in extracellular vesicles).

Thus the invention provides a gene delivery vehicle for use according the invention, wherein the miRNA is under control of a relatively weak promoter, preferably a promoter selected from a Polymerase II promoter, a chicken-beta actin promoter, an EF1alpha promoter, a PGK promoter or a suitable tissue-specific promoter, which are well known in the art.

The gene delivery vehicles according to the invention are intended for the treatment of various diseases in (human) subjects, but may also be used to generate extracellular vesicles in vitro, whereby said extracellular vesicles produced become gene delivery vehicles according to the invention themselves. To achieve this a composition comprising the original gene delivery vehicle may be provided to a cell culture. The cell culture is then transduced with the gene delivery vehicle. Using a gene delivery vehicle according to the invention will result in the transduced cell producing extracellular vesicles, in particular exosomes and/or microvesicles that can be isolated and used to produce further gene delivery vehicles or be used in a pharmaceutical composition to be administered to a subject.

The invention therefore provides a composition comprising a gene delivery vehicle according to the invention and suitable excipients, such as buffers and stabilizers, antioxidant etc. In one particular embodiment these compositions are used to transduce cells in vitro or ex vivo, in which case the excipients will need to be compatible with cell culture. In other preferred embodiments the compositions are used for treatment of (human) subjects. For that purpose the invention provides a pharmaceutical composition comprising a gene delivery vehicle according to the invention and suitable excipients for pharmaceuticals. In the case of AAV gene delivery vehicles a pharmaceutical composition typically comprise physiological buffers, such as e.g. PBS, comprising further stabilizing agents such as e.g. sucrose. Such compositions are compatible with and suitable and intended for use in subsequent intravenous, intrathecal, intraparenchymal, intravitreal, subretinal administration or use in organ-targeted vascular delivery such as intraportal or intracoronary delivery or isolated limb perfusion.

Dosing of the gene delivery vehicles will typically be determined in rising dose studies in animals. It is appreciated that doses of AAV gene delivery vehicles may vary depending on the different routes of administration.

### EMBODIMENTS

1. A gene delivery vehicle for use in delivery of a miRNA to a target cell resulting in silencing of a desired gene in said transduced target cell, whereby spread of said miRNA to other non-transduced target cells results in silencing of said desired gene in said non transduced target cells.

2. A gene delivery vehicle for use in delivery of a miRNA to a target cell resulting in silencing of a desired gene in said transduced target cell and spread of said miRNA to other target cells for silencing of said desired gene, wherein said gene delivery vehicle comprises a miRNA scaffold.

3. A gene delivery vehicle for use in delivery of a miRNA to a target cell resulting in silencing of a desired gene in said transduced target cell and spread of said miRNA to other target cells for silencing of said desired gene, wherein said gene delivery vehicle comprises a mir-451 scaffold or a functional equivalent thereof.

4. A gene delivery vehicle for use according to any one of embodiments 1-3, whereby the spread occurs through packaging of the miRNA in extracellular vesicles.

5. A gene delivery vehicle for use according to embodiment 4, whereby said extracellular vesicles are exosomes or microvesicles.

6. A gene delivery vehicle for use according to anyone of embodiments 1-5, wherein said silencing comprises nuclear silencing.

7. A gene delivery vehicle for use according to any one of embodiments 1-6, wherein said silencing comprises cytoplasmic silencing.

8. A gene delivery vehicle for use according to any one of embodiments 1-7, wherein said gene delivery vehicle is a virus derived particle.

9. A gene delivery vehicle for use according to any one of embodiments 8, wherein said gene delivery vehicle is an AAV based particle.

10. A gene delivery vehicle for use according to any one of embodiments 1-9, for the treatment of neurodegenerative diseases.

11. A gene delivery vehicle for use according to 10 for the treatment of a neurodegenerative disease selected from the group consisting of Huntington's disease, amyotrophic lateral sclerosis (ALS), spinocerebellar ataxias, Parkinson's disease, Alzheimer's disease, and frontotemporal dementia (FTD).

12. A gene delivery vehicle for use in delivery of a miRNA preferentially to a brain cell resulting in silencing of a desired gene and spread of said miRNA to other brain cells for silencing of said desired gene, wherein said gene delivery vehicle comprises a mir-451 scaffold or a functional equivalent thereof.

13. A gene delivery vehicle for use according to any one of embodiments 1-9, for the treatment of liver diseases or metabolic disorders.

14. A gene delivery vehicle for use in delivery of a miRNA preferentially to a liver cell resulting in silencing of a desired gene and spread of said miRNA to other liver cells for silencing of said desired gene, wherein said gene delivery vehicle comprises a mir-451 scaffold or a functional equivalent thereof.

15. A gene delivery vehicle for use according to embodiment 13 or embodiment 14 wherein the miRNA is under control of a relatively weak promoter, such as a promoter is selected from the group consisting of a Polymerase II promotor, a chicken-beta actin promoter, an EF1alpha promoter, a CAG promoter, a PGK promoter or a tissue-specific promoter for liver expression such as LP1, or AAT.

16. A gene delivery vehicle for use in delivery of a miRNA preferentially to a heart cell resulting in silencing of a desired gene and spread of said miRNA to other heart cells for silencing of said desired gene, wherein said gene delivery vehicle comprises a mir-451 scaffold or a functional equivalent thereof.

17. A gene delivery vehicle for use in delivery of a miRNA preferentially to a cell in the eye resulting in silencing of a desired gene and spread of said miRNA to other cells in the eye for silencing of said desired gene, wherein said gene delivery vehicle comprises a mir-451 scaffold or a functional equivalent thereof.

18. A gene delivery vehicle for use in delivery of a miRNA preferentially to a muscle cell resulting in silencing of a desired gene and spread of said miRNA to other muscle cells for silencing of said desired gene, wherein said gene delivery vehicle comprises a mir-mir-451 scaffold or a functional equivalent thereof.

19. A gene delivery vehicle for use according to any one of embodiments 1-12 and 16-18, wherein the miRNA is under control of a relatively weak promoter.

20. A gene delivery vehicle for use according to embodiment 19, wherein the promoter is selected from a Polymerase II promotor, a chicken-beta actin promoter, a CAG promoter, an EF1alpha promoter, a PGK promoter or a tissue-specific promoter.

21. A composition comprising a gene delivery vehicle according to any one of embodiments 1-20 and suitable excipients.

22. A pharmaceutical composition comprising a gene delivery vehicle according to any one of embodiments 1-21 and suitable excipients.

23. A pharmaceutical composition according to embodiment 22 for use in intravenous, intrathecal, intraparenchymal, intravitreal, subretinal administration.

24. A pharmaceutical composition according to embodiment 23 for use in, organ-targeted vascular delivery such as intraportal or intracoronary delivery or isolated limb perfusion.

The invention will be explained in more detail in the following experimental section.

### EXAMPLES

### Example 1

Material and Methods

Expression Cassettes, miRNAs and AAV Vectors

Expression cassettes and AAV vectors used in the studies are as described i.a. in WO2016102664 and Miniarikova et al., 2016. The expression cassette was inserted into a vector genome backbone flanked by two intact non-coding inverted terminal repeats (ITR) that originate from AAV2. Briefly, miRNA expression cassettes comprise the chimeric chicken-beta actin promoter, the miRNA sequence was replaced by a sequence designed to target a selected gene sequence and engineered in the pri-mir-451 backbone, and the human growth hormone polyA signal (Schematically depicted in FIG. 1). The sequence of an exemplary expression cassette is depicted in FIG. 2. The 22 nucleotide sequence targeting ATXN-3 was 5′-TCTGGAACTACCTTGCATACTT-3′ (SEQ ID NO. 2; 22 nts) and is comprised in the sequence depicted in FIG. 2. The sequence of an exemplary expression cassette is depicted in FIG. 2. The 22 nucleotide sequence targeting the Huntington gene sequence that was used in these experiments corresponds with 5′-AAGGACTTGAGGGACTCGAAGA-3′ (SEQ ID NO. 3; 22 nts).

The sequence targeting the Huntington gene sequence corresponds with the H12 candidate as described in WO2016102664 and Miniarikova et al., 2016, which is incorporated herein by reference. The sequences selected targeting the ATXN3 and HTT genes represent sequences that, when expressed, and processed by the RNAi machinery, are complementary to target sequences in mRNAs expressed from the ATXN3 and HTT genes, respectively. Hence, the RNA sequences that are complementary to HTT and ATNX3, respectively, when comprised in a miRNA scaffold as depicted e.g. in FIG. 1, correspond respectively with 5′-AAGGACUUGAGGGACUCGAAGA-3′ (SEQ ID NO. 5; 22 nts), the sequence that was used to target ATXN-3 was 5′-UCUGGAACUACCUUGCAUACUU-3′ (SEQ ID NO. 6; 22 nts). AAV vectors used in these studies were based on the AAV5 serotype and manufactured using insect cell based manufacturing. Briefly, Recombinant AAV5 harbouring the expression cassettes were produced by infecting SF+ insect cells (Protein Sciences Corporation, Meriden, Conn., USA) as described (Lubelski et al. Bioprocessing Journal, 2015). Following standard protein purification procedures on a fast protein liquid chromatography system (AKTA Explorer, GE Healthcare, Chicago, Ill., USA) using AVB sepharose (GE Healthcare, Chicaco, Ill., USA), the titer of the purified AAV was determined using qPCR.

Small RNAseq analysis of the mature miRNA sequences produced from the 451 scaffold targeting the Huntington gene (in order of abundance, expressed as %) are listed below, (miHTT). The sequences listed correspond with RNA sequences, i.e. the sequences listed below are DNA sequences, which, when the Ts are replaced with Us, represents the corresponding RNA sequences.

| SEQ | | | |
|---|---|---|---|
| ID | SEQUENCE | | % |
| NO. | (5′-NNN-3′) | length | reads |
| 7 | AAGGACTTGAGGGACTCGAAGACG | 24 | 45.7 |
| 8 | AAGGACTTGAGGGACTCGAAGAC | 30 | 16.0 |
| | GAGTCCC | | |
| 9 | AAGGACTTGAGGGACTCGAAGAC | 23 | 11.5 |
| 3 | AAGGACTTGAGGGACTCGAAGA | 22 | 8.3 |
| 10 | AAGGACTTGAGGGACTCGAAGACGA | 25 | 4.9 |
| 11 | AAGGACTTGAGGGACTCGAAG | 21 | 3.1 |
| 12 | AAGGACTTGAGGGACTCGAAGACGAGT | 27 | 2.3 |
| 13 | AAGGACTTGAGGGACTCGA | 19 | 1.6 |
| 14 | AAGGACTTGAGGGACTCGAAGACGAGT | 31 | 1.1 |
| | CCCT | | |
| 15 | AAGGACTTGAGGGACTCGAA | 20 | 1.0 |

Small RNAseq analysis of the mature miRNA sequences produced from the 451 scaffold targeting the ATXN3 gene (in order of abundance, expressed as %) are listed below (miATXN). The sequences listed correspond with RNA sequences, i.e. the sequences listed below are DNA sequences, which, when the Ts are replaced with Us, represents the corresponding RNA sequences.

| SEQ | | | |
|---|---|---|---|
| ID | SEQUENCE | | % |
| NO. | (5′-NNN-3′) | length | reads |
| 2 | TCTGGAACTACCTTGCATACTT | 22 | 53.0 |
| 16 | TCTGGAACTACCTTGCATACTTAT | 24 | 18.0 |
| 17 | TCTGGAACTACCTTGCATACTTA | 23 | 11.0 |
| 18 | TCTGGAACTACCTTGCATACT | 21 | 6.1 |
| 19 | TCTGGAACTACCTTGCATACT | 30 | 2.9 |
| | TATGCAAGG | | |

The most abundant sequences were used for the analysis of extracellular vesicles (EV), i.e. the 24 nts sequence corresponding SEQ ID NO.7 was used in the analysis representing miRNA found in EV, likewise, the sequence corresponding with SEQ ID NO.2 was used in the analysis representing miRNA found in EV. For analysis of brain samples, for both ATXN3 and HTT the 22 nucleotide sequences were used in the analysis.

Differentiation of Forebrain Neuronal Cultures from Human HD Induced Pluripotent Stem Cells (iPSCs)

HD iPSCs (ND42229*B) containing 71 CAG repeats were ordered from (Coriell Institute Stem Biobank, passage 25). These cells were generated from human HD fibroblasts GM04281 (Coriell Institute Stem Biobank) reprogrammed with six factors (OCT4, SOX2, KLF4, LMYC, LIN28, shRNA to P53) using episomal vectors. iPSCs were maintained on matrigel coating with mTeSR medium for several passages, following the manufacturer's instructions (StemCell Technologies, Vancouver, Canada). Non-differentiated colonies were released using ReLeSR reagent during each passage and diluted 1:5-20 (StemCell Technologies). For the neural induction, cells were plated onto AggreWell™ 800 plate at day 0 as 3×106 cells per well in STEMdiff™ Neural Induction Medium (StemCell Technologies). At day 5, embryoid bodies were formed and replated onto poly-D-lysine/laminin coated 6-well plates. Coating was prepared with poly-D-lysine hydrobromide (0.1 mg/mL) and Laminin from Engelbreth-Holm-Swarm murine (0.1 mg/ml) (Sigma-Aldrich). At day 12, the neuronal rosettes were selected using STEMdiff™ Neural Rosette Selection Reagent (StemCell Technologies) and replated in poly-D-lysine/laminin coated plates. Next day, differentiation of neural progenitor cells was initiated using STEMdiff™ Neuron Differentiation Kit (StemCell Technologies). From day 19, cells were matured using STEMdiff™ Neuron Maturation Kit for a minimum of two weeks (StemCell Technologies). A typical example of differentiated cells is shown in FIG. 10.

Extracellular Vesicles (EVs) Precipitation and Detection of miRNA Secreted by iPSC-Derived Neurons

Cells were transduced with different doses of AA5-miHTT (3E11gc, 3E12gc and 3E13gc) at MOI E5, E6 and E7 or AAV5-miATXN (3E12gc and 3E13gc) at MOI E6 and E7. Medium from transduced neuronal cultures was refreshed every two days and collected on day 5 and day 12 after transduction, and centrifuged at 3000×g for 15 min to remove cells and cell debris. The EVs were isolated with ExoQuick-TC (System Bioscience, California, USA) according to manufacturer's protocol. 3 ml of ExoQuick buffer was added to 10 ml of conditioned medium and incubated at 4° C. overnight. Next day, the exosomes were collected at 1500×g for 30 minutes and the supernatant was discarded. The residual solution was additionally centrifuged at 1500×g for 10 minutes. The EVs pellets were re-suspended in appropriate buffers and stored at −80° C. for further experiments.

RNA Isolation and Real-Time qPCR

RNA was isolated from cells using Trizol according to the manufacturer's protocol (Invitrogen). To measure mRNA knock-down, cDNA was generated using Maxima Synthesis Kit (Thermo Scientific). cDNA was analyzed by qPCR using commercially available primers and probes: human HTT (assay ID Hs00918134_m1) and human GAPDH (assay ID Hs02758991_g1) (corresponding primer and probes available using assay IDs at Thermo Scientific). To detect miHTT expression levels, RT-qPCR was performed using TaqMan Fast Universal kit (Thermo Scientific), and commercially available primers and probes hsa-miR-16 (000391, Applied Biosystems). Custom-made miHTT primers and probes (assay ID CTXGPY4, Thermo Scientific) and miATXN primers and probes (assay ID CTEPRZE, Thermo Scientific), were used for SEQ ID NOs. 7 and 2, respectively. The expression level was normalized to hsa-miR-16 levels. Fold changes of miRNA expression were calculated based on 2\hat{ }ΔΔCT method (Livak and Schmittgen, Method. Methods. 2001, December; 25(4):402-8). miRNA expression was calculated based on a standard line with synthetic RNA oligos. For the viral vector DNA isolation, neuronal cultures were processed using DNeasy Blood & Tissue Kit (Qiagen, Valencia, Calif., USA) following manufacturer's protocol. AAV5 vector genome copies were measured by qPCR reaction using SYBR Green protocol (Applied Biosystems, Foster City, Calif., USA) and validated standard line for detection of CAG promoter. Forward primer sequence: GAGCCGCAGCCATTGC and reverse primer sequence: CACAGATTTGGGACAAAGGAAGT (SEQ ID NO.25-26). The standard line was used to calculate the genome copies per DNA microgram.

Protein Isolation and Western Blotting

EVs precipitates were lysed using RIPA lysis buffer (Sigma-Aldrich) supplemented with protein inhibitor cocktail (cOmplete™ ULTRA Tablet; Roche, Basel, Switzerland). Total protein concentration was quantified using a Bradford Protein Assay (Bio-Rad, Hercules, Calif., USA) and absorbance was measured at 600 nm on the GloMax Discover System (Promega). Equal amounts of sample protein (10-30 μg) were incubated with β-mercaptoethanol and Laemmli buffer at 95° C. for 5 min and separated using 4-20% Mini-Protean TGX Stain-Free Protein Gel (Bio-Rad). Samples were transferred to PVDF membranes by Trans-Blot Turbo Transfer system (Bio-Rad) using the “Mixed MW” protocol. Blot was incubated with 3% Blotting-Grade blocker (Bio-Rad) in 1×Tris Buffered Saline (TBS) for 1 hour at room temperature, followed by immunoblotting with selected primary antibody overnight at 4° C. (see Table 1 below). Chromogenic signals were detected after 2 hours incubation with HRP-conjugated secondary antibodies (see table below) and 5 min incubation with SuperSignal Pico sensitivity Substrate (ThermoScientific) using ChemiDoc Touch Gel Imaging System (Bio-Rad).

TABLE 1
Antibodies used for Western Blotting

| | Antibodies | Brand | Ordering number | Concentration |
|---|---|---|---|---|
| EVs | CD63 | System Biosciences | EXOAB-CD63A-1 | 1:1000 |
| | Alix | Abcam | ab76608 | 1:1000 |
| | TSG101 | Abcam | ab30871 | 1:1000 |
| ER | Calnexin | Abcam | ab92573 | 1:20000 |
| Mitochondria | ATPase | Abcam | ab58475 | 1:1000 |
| RISC | Ago2 | EMD Millipore | 07-590-25UG | 1:1000 |
| Cytoplasm | a-tubulin | Abcam | ab7291 | 1:1000 |
| | [DM1A] | | | |
| Secondary | HRP goat | Abcam | ab97051 | 1:20000 |
| antibodies | anti-rabbit | | | |
| | HRP rabbit | DAKO | | 1:20000 |
| | anti-mouse | | | |

Results

Detection of miRNAs in the Medium from AAV-Transduced iPSC-Derived Neurons after 5 Ad 12 Days.

HD iPSC-derived neuronal cells were transduced with two therapeutic viral vectors: AAV5-miHTT and AAV5-miATXN. Results show a dose-dependent transduction of HD-iPSC derived neuronal cells with three doses of AAV5-miHTT (3E11gc, 3E12gc and 3E13gc) and two doses of AAV5-miATXN (3E12gc and 3E13gc) at 20 days after transduction (FIG. 3). After 5 and 12 days, both miHTT and miATXN molecules were detected in a dose-dependent manner in the EVs precipitated from the medium of neuronal cells. Graphs depicted in FIGS. 4 and 5 represent fold change expression of miHTT and miATXN, respectively, normalized to endogenous miR-16 and compared to control cells.

EV-miHTT Secreted from Transduced Cells Strongly Correlates with AAV Dose and miHTT Expression in the Cells

We summarized data from independent experiments showing a robust correlation between secreted miHTT molecules detected in the medium of transduced cells and both viral vector dose (FIG. 6A) and miHTT expression within the cells (FIG. 6B) (n=6). Experiments were performed separately at different time points. Graphs represent AAV dose (log 10) and relative expression of miHTT detected by qPCR. Each dot represents the average of an independent experiment which was performed in duplicate or triplicate.

Characterization of EVs Precipitated from Medium of iPS-Derived Neurons by Western Blot

In order to confirm the composition of EVs precipitated from the medium, protein was isolated and analyzed by western blot with different markers (See Methods section) (FIG. 7). Results confirmed a presence of EV and exosomal markers (CD63, Alix and TSG-101), microvesicles (Calnexin) and RISC complex (Ago2) to which functional miRNAs are bound to. Cellular markers (a-tubulin and ATPase) were used as controls to confirm the absence of cells or cellular debris.

Therapeutic miRNA Molecules Secreted from AAV5-Transduced Neuronal Cells can be Transferred to Naïve Neuronal Cultures in a Dose-Dependent Manner.

Medium from PBS, AAV5-miHTT and AAV5-miATXN transduced iPSC-derived neuronal cells was collected and EVs precipitated by Exoquick (see Methods section). EVs pellets were pooled together and added in different concentrations (0.1×, 0.5× 1×, 2× or 5×) to 1E5 naive iPSC-derived neuronal cells in one well of a 24-well plate (see FIG. 8A). Cells were harvested 24 hours after EV-transfer. Results depicted in FIGS. 8B and 8C indicate a dose-dependent transfer of miHTT and miATXN to naïve neuronal cells.

Non-Viral Vector Functional Transfer of Therapeutic miRNAs Between Neuronal Cells.

iPS-derived neuronal cells transduced with 3E13 gc AAV5-miHTT from the previous experiment were seeded in Corning® Transwell® polyester membrane cell culture inserts (24 mm, 0.4 μm pore) (Sigma). Naïve iPS-derived neurons were seeded 5E5 neurons/well in a 6-well plate. 48 hours after seeding, inserts were placed on top of the wells and cells were cocultured for 2 weeks, refreshing medium every 2 or 3 days (n=6). Naïve iPSC-derived cells without inserts were used as controls. Cells were harvested separately with Accutase (STEMcell technologies) and divided for different molecular analysis including AAV5 genome copies (FIG. 9A) and HTT mRNA lowering (FIG. 9B) by real-time qPCR (see Methods section). Results show that there was no viral vector genome transfer between donor and recipient cells since only donor cells in Transwell inserts contained high levels of AAV5 genome copies detected by qPCR (n=6). Interestingly, in both donor transduced cells and recipient naïve cells we detected a 30% and 20% HTT mRNA lowering, respectively, compared to control cells (n=6). This preliminary data indicates a functional transfer of therapeutic miRNAs between neuronal cells.

Conclusions

To conclude, therapeutic miRNA molecules are present in a dose-dependent manner in the EVs precipitated from the supernatant of HD neuronal cultures treated with AAV5-miHTT-451 and AAV5-miATXN-451 gene therapies. Extracellular therapeutic miRNAs levels secreted from transduced cells strongly correlate with AAV5 dose and miRNA expression in the cells. Therapeutic miRNA molecules within EVs secreted from AAV5-transduced neuronal cells can be transferred to naïve HD neuronal cultures in a dose-dependent manner. Combined this data indicated viral vector expressed therapeutic miRNAs can be functionally transferred between neuronal cells.

### Example 2

This example assessed AAV5-miHTT vector DNA levels and miHTT-24nt microRNA expression targeting mutant HTT gene in different brain regions of non-human primates (cynomolgus monkeys) at 6 months post-AAV5-miHTT intraparenchymal administration in caudate and putamen.

Study Design

This study in male and female cynomolgus monkeys employs a gene therapy product designed to silence the expression of huntingtin by means of the RNA interference (RNAi) mechanism using a engineered microRNA (miHTT) targeting human huntingtin, delivered via adeno-associated viral vector serotype 5 (AAV5-miHTT) in the brain. The design of the study is outlined table below.

| | | Dose level (gc/brain) and volume | | | |
|---|---|---|---|---|---|
| | | (in uL Per target region) | | Number of animals | |
| Group | Treatment | Dose level | Volume | Male | Female |
| 1 | Control | 0 (PBS) | 4 × 100 | 3 | 3 |
| 2 | Low | 2E12 | 4 × 100 | 3 | 3 |
| 3 | Mid | 7E12 | 4 × 100 | 3 | 3 |
| 4 | High | 2E13 | 4 × 100 | 3 | 3 |

Methods

Animals were injected with AAV5-miHTT at different doses as indicated in the table with study design. AAV5-miHTT was delivered bilaterally in the brain, at the doses indicated, directly in the caudate and putamen (100 uL per region), by MRI-guided convention-enhanced delivery (CED). Six months after administration, necropsy was performed and throughout the brain punches were taken of approximately 3 mm3 for biomolecular analysis. Vector DNA levels were analyzed by QPCR using specific primers directed against the AAV5-miHTT vector (primer A (forward) 5′CCCACCAGCCTTGTCCTAAT 3′ (SEQ ID NO. 22); primer B (reverse) 5′GTTCCTCAGATCAGCTTGCAT 3′ (SEQ ID NO. 23); probe C (5′ 6FAM)ACGGGCCCGTCGACTGCAGAGGC (SEQ ID NO. 24; all Invitrogen) and miHTT expression levels analyzed by RT-QPCR (assay ID CTXGPY4, Thermo Scientific) using standard methods.

Results

Vector copy numbers in the injected striatal areas (average of caudate and putamen) were around 3.6E+07 gc/μg DNA in the low dose group, 7.6E+07 copies/μg DNA in the mid dose group and 1.5E+08 gc/μg DNA in the high dose group. The cerebral cortex (frontal, temporal and motor) showed average vector levels of around 3.1E+05, 2.2E+06 and 5.7E+06 gc/μg DNA, for the low, mid and high dose groups, respectively. miHTT expression levels were assessed in a two-step RT-qPCR assay using 10 ng of RNA sample from selected Cynomolgus monkey brain sections and off-target tissues. For AAV5-miHTT dosed groups, miHTT expression correlated with vector DNA levels, with the highest concentrations detected in the injected areas, and spread to other striatal structures (globus pallidus) and to cortical areas (results depicted in FIGS. 12 and 13).

Conclusions

Six months after AAV5-miHTT gene therapy delivered locally in the striatum (caudate and putamen) of cynomolgus monkeys, high levels of vector DNA are detected in target areas (striatum), and in areas further away from the injected areas (e.g. cerebral cortex). Similarly, miHTT expression is relatively high, not only in target areas, but also in areas further away from the injection site. The levels of miHTT correlate with the levels of vector DNA, but are proportionally relatively higher in areas further away from the injection site than the vector DNA levels. Furthermore, one can observe a striking absence of a dose-response for miHTT in regions further away from the injection site which concomitantly show a strong dose response when assessing vector DNA levels in these regions. This is suggestive of non-viral vector mediated spread of miHTT, such as via extracellular vesicles or exosomes/microvesicles, from injected to non-injected brain areas, masking the dose response resulting from transgene expression in those areas, and explaining the absence of a dose response for miHTT and the higher than expected miHTT levels in regions further away from the injection site.

### Example 3

In Vitro Data Indicates Functional Transfer of Therapeutic miRNAs Mediated by Extracellular Vesicles

This example shows that neuronally secreted therapeutic miRNAs are functionally transferred to recipient cells.

Materials and Methods

EVs and Protein Species Purification by SEC

iPSC-derived forebrain neuronal cells (Example 1) were transduced with AAV5-miHTT (3E13gc) at MOI E7 or AAV5-miATXN3 (3E13gc) at MOI E7. Medium from transduced cultures was collected and centrifuged at 4000×g for 15 min to remove cell and cell debris. Separation of EVs from protein species was achieved by size-exclusion chromatography (SEC) with the commercial qEV10 columns, 70 nm (Izon). Briefly, after washing the column with PBS, 10 ml of medium was loaded into the column and 21 fractions of 5 ml were collected. Every fraction was then concentrated to 200-300 ul by Amicon Ultra-15 Centrifugal Filter Units (10 kDa MWCO) by centrifugation at 4000×g for 15 min at 4° C. RNA isolation of every fraction was performed with Direct-zol RNA kit according to manufacturer's protocol (Zymo research).

iPSC-Derived Neurons and Fibroblast Cultures

Three-week matured iPSC-derived forebrain neurons from HD patient (Example 1) were plated in 4-well chamber slides in BrainPhys medium (StemCell technologies). Neuronal cells were transduced with a high dose of AAV-miATXN3 (1E12gc/well) at MOI 2E7. Five days after neuronal transduction, neuronal cells were properly washed, wells removed, and slides were placed into a petri dish (150 mm) for co-culturing with non-transduced fibroblast cells. Fibroblast derived from human control individuals were purchased at Coriell repository. Cells were plated in 4 well chamber slides in MEM medium (Thermo Fisher) supplemented with 2 mM L-Glutamine, 15% Fetal Bovine Serum and 1% Penicillin/Streptomycin.

Co-culture system comprised of 3× transduced neuron slides and 1× non-transduced fibroblast slide (ratio 3:1) in MEM medium for 8 days. As a control, 3× non-transduced neurons slides were co-cultured with 1× non-transduced fibroblast slide in MEM medium. As a positive control, fibroblast cells were directly transduced with two doses of AAV-miATXN3 at MOI 1E6 and 1E8 and maintained for 8 days, likewise to the co-culture system (see FIG. 14). Cells were washed, harvested separately and resuspend in 200 ul Trizol for RNA purposes, or in 100 ul RIPA buffer for protein assays, and stored at −80 C.

Results 1—Secreted AAV-Delivered Therapeutic miRNAs are Associated with Both EVs and Protein Complexes In Vitro

Endogenous miRNAs have been found to circulate extracellularly in association with not only EVs but also protein species such as high-density lipoproteins (HDLP) and Ago2 complexes (Arroyo, et al., March 2011 PNAS, pnas.1019055108; Yuana et al., (2014) Journal of Extracellular Vesicles, 3(1), 1-5). EV isolation methods based on precipitation (as ExoQuick) give a higher RNA yield but often result in the co-isolation of non-vesicular protein species. In order to achieve a better enrichment and separation of EVs from other co-isolated proteins, size exclusion chromatography (SEC) columns were used followed by centrifugation-based concentration (Boing et al., (2014) Journal of Extracellular Vesicles, 3(1), 1-11) (FIG. 13A). According to manufacturer's protocol fractions 6-8 are typically enriched in EVs (70-110 nm), and fractions 14-17 in proteins, including HDLP (<70 nm). The aim of this experiment was to confirm the association of secreted therapeutic miRNAs with EVs released from AAV-transduced neuronal cells. For this purpose, the presence of two artificial therapeutic miRNAs (miHTT and miATXN3) in the different SEC fractions isolated from the medium of transduced iPSC-derived neurons was investigated. Results showed that both AAV-delivered therapeutic miRNAs were enriched in the EV-containing fractions from SEC as well as in the fractions containing the bulk of the proteins, protein complexes, and EVs with a diameter <70 nm (the so-called “protein fraction”) when secreted to the extracellular medium by neuronal cells (FIG. 13C-D). This protein fraction can contain high density lipoproteins (HDLP), other protein complexes (like Ago2) and small EVs. As a control, the abundance of an endogenous miRNA (miR-16) was measured, which is known to be associated with proteins, but not enriched in EVs (FIG. 13B). These results confirm the association of AAV-delivered miR-451-based therapeutic miRNAs with EVs in vitro.

Results 2—Continuous Transfer of Secreted Therapeutic miRNAs Results in Lowering of Gene Expression in Recipient Cells

To investigate whether therapeutic miRNAs are still functional after being taken up by recipient cells, AAV-miATXN3 transduced “secreting” cells and non-transduced “recipient” cells were co-cultured together (FIG. 14). In order to reduce possible AAV-contamination, fibroblast cells were selected as recipient cells due to their low AAV-transduction efficiency. iPSC-derived neurons were selected as secreting cells. AAV-miATXN3 transduced neurons and non-transduced fibroblast were co-cultured together for 8 days in a ratio 3:1. Results showed that neuronal cells were efficiently transduced with AAV5, but also fibroblast cells co-cultured together contained high number of viral genome copies (FIG. 15A). Unexpectedly, very high numbers of viral genome copies were detected in fibroblasts directly transduced with AAV5-miATXN3, i.e, ten times higher gc in fibroblasts transduced with MOI 1E6 compared to neurons transduced with higher dose MOI 2E7. Since fibroblasts were cultured less days and not properly washed, it is likely that the high number of genome copies was due to viral particle contamination. Then, miATXN3 abundance was measured by TaqMan qPCR (FIG. 15B). Consistent with the efficient transduction of neuronal cells, very high levels of mature miATXN3 molecules were detected in neurons transduced with AAV-miATXN3. However, much lower number of miATXN3 molecules were detected in fibroblasts directly transduced, indicating that even if the virus entered the cells, the expression of the artificial miRNA in these cells is very low, even after 5 days. Similar levels of miATXN3 molecules were detected in fibroblast cells co-cultured with transduced neurons. It is likely that viral contamination contributed to some extent to the expression of miRNA in the recipient non-transduced fibroblast cells. To investigate the efficacy of miATXN3 to lower the expression of ATXN3 mRNA, the relative expression of ATXN3 normalized to housekeeping gene GAPDH was compared to naïve cells of the same cell type (FIG. 15C). Consistent with the high expression of miATXN3 molecules in neuronal cells a 45% lowering of ATXN3 was measured, indicating that miATXN3 is functional and capable of lowering ATXN3 in the secreting neuronal cells. In fibroblast cells co-cultured together with transduced neurons, the transfer of miATXN3 resulted in 20% lowering of ATXN3. On the contrary, only a subtle or no lowering was detected in fibroblast directly transduced with AAV, which contained similar levels of miATXN3 molecules. It was concluded that while there was some viral contamination, the ATXN3 lowering observed in recipient cells is, to some extent, due to non-viral transfer of miATXN3 molecules secreted from neuronal cells and taken up by recipient fibroblast cells.

### Example 4

Widespread Mutant HTT Protein Brain Lowering in tgHD Minipigs at 6- and 12-Months Post-Administration of AAV5-miHTT in Striatum

In this study, AAV5-miHTT vector DNA levels, miHTT-24nt microRNA expression and mutant HTT protein lowering were assessed in several brain regions of tgHD minipigs at 6 and 12 months post-AAV5-miHTT intraparenchymal administration in caudate and putamen (target regions).

Animals

All experiments were carried out according to the guidelines for the care and use of experimental animals and approved by the State Veterinary Administration of the Czech Republic. TgHD minipigs (Baxa et al., J Huntingtons Dis. 2013; 2(1):47-68) and healthy controls of both sexes, 6 months old, were used.

Study Design

The whole study consisted of n=15 six months old AAV5-miHTT treated and n=15 naïve (untreated) tgHD minipigs. Animals were divided in three interim sacrifice timepoints post-treatment: after 6 months (n=3/group), 12 months (n=4/group) or >2 years (n=8/group). At this stage, the interim sacrifices of 6 and 12 months have been performed, and the animals planned for sacrifice beyond 2 years post-treatment are still in the in-life phase. More specific information on the study design can be found in Table X1.

TABLE XI
Study design

| | | | | Observation | Age at sacrifice |
|---|---|---|---|---|---|
| Treatment | N | Dose* | Animal number (M/F) | period | (months) |
| AAV5- | 3 | 1.2 × 10¹³ gc | T29 (F), T31 (F), T34 (M) | 6 months | 13, 12, 14 |
| miHTT | | total | | | |
| Naive | 3 | No treatment | T37 (F), T65 (F), T66 (F) | 6 months | 14, 10, 10 |
| AAV5- | 4 | 1.2 × 10¹³ gc | T55 (F), T58 (F), T63 (F), | 12 months | 17, 17, 16, 16 |
| miHTT | | total | T62 (F) | | |
| Naive | 4 | No treatment | T68 (F), T77 (F), | 12 months | 17, 17, 16, 15 |
| | | | T81 (F), T84 (M) | | |
| AAV5- | 8 | 1.2 × 10¹³ gc | T38 (F), T39 (M), T45 (F), | ≥2 years | N/A |
| miHTT | | total | T51 (F), T70 (F), T73 (F), | | |
| | | | T87 (M), T93 (F) | | |
| Naive | 8 | No treatment | T56 (F), T85 (F), | ≥2 years | N/A |
| | | | T100 (F), T101 (F), | | |
| | | | T91 (F), T140 (F), | | |
| | | | T125 (M), T129 (F) | | |

*Bilateral 2 × 100 μl (Putamen and Caudate) of 3 × 10¹³ gc/ml injection (400 μl total), by MRI-guided convention-enhanced-delivery (CED). F: female, M: male

The animals were injected bilaterally into the caudate and putamen with a total of four catheters, one in each putamen and each caudate. Each minipig received 100 μl of 1.2×10¹³ gc/mL AAV-miHTT per catheter using the Renishaw drug delivery system. At 6 months after injection, n=3 treated animals (T9, T31, T43) and n=3 naïve (untreated) controls (T37, T65, T66) were sacrificed. At 12 months after injection, n=4 treated animals (T55, T58, T62, T63) and n=4 naïve (untreated) controls (T68, T77, T81, T84) were sacrificed. From each of these animals, the brains were collected and sliced coronally (4 mm-thick sections) after which a total of 54 (6 months sacrifice) or 170 (12 months sacrifice) brain punches of 3 mm in diameter were taken bilaterally. Each punch of the left hemisphere (even numbers) was divided in four parts for different purposes (DNA, RNA, protein or backup), while the punches from the right hemisphere (odd numbers) were not divided and kept as a backup. All punches were kept at −80 C until further analysis. The brain punch dissection scheme is depicted in FIG. 16.

Materials and Methods

VECTOR—AAV5 vector encoding cDNA of the miHTT cassette was packaged into AAV5 by a baculovirus-based AAV production system (uniQure, Amsterdam, The Netherlands) as previously described (Evers et al., Mol Ther. 2018 Sep. 5; 26(9):2163-2177).

DNA Isolation and Determination of Vector Genome Copies

Tissue punches were crushed using CryoPrep System (Covaris, Woburn, Mass.) and powder was divided for RNA and DNA analyses. For DNA isolation, 20 mg of powdered tissue was used with the DNeasy 96 Blood and Tissue kit (Qiagen, Germany). Primers specific for the CAG promoter were used to amplify a sequence specific for the transgenes by SYBR Green Fast qPCR (Thermo Fisher Scientific). The amount of vector DNA was calculated from a plasmid standard curve, which was taken along on the same plate. Results were reported as gc per μg of genomic DNA.

RNA Isolation and RT-QPCR

For RNA isolation, crushed tissue was homogenized in TRIzol using the Fast-Prep-24™ 5 G at a speed of 6 m/s for 40 seconds, followed by total RNA extraction with Direct-zol™ RNA MiniPrep kit (Zymo Research). To examine miHTT RNA expression, cDNA was synthesized from isolated total RNA with gene-specific RT primers targeting miHTT using TaqMan MicroRNA Reverse Transcription Kit (Applied Biosystems). A single stranded miHTT RNA standard line was taken along. Next, gene-specific TaqMan qPCR was performed with miHTT-specific probes using TaqMan Fast Universal PCR Master Mix (Applied Biosystems). Using the miHTT standard line, miHTT molecules per reaction were determined. Subsequently, the amount of miHTT molecules per cell of tissue were calculated based on the assumption that one cell contains 15 pg total RNA.

Mutant HTT Protein Immunoassay

Crushed tissues were homogenized in 200 μL of 1× lysis buffer (1×PBS, 0.4% Triton X, Protease inhibitor) in Fastprep96 homogenizer (MP Biomedicals; 3×30 sec cycles at 1600 rpm), and stored overnight at −80° C. Samples were then centrifuged for 10 minutes at 14000 rpm and supernatants were transferred to 96-well plates. Total protein concentration was determined by BCA assay using 2 μL of homogenate diluted 10-fold in lysis buffer (in triplicate) as per manufacturer's instruction. An ultrasensitive single molecule counting (SMC) immunoassay was carried out on the homogenized tissues and CSFs. The assay is based on the use of a capture antibody, coated on magnetic beads, and a detection antibody labelled with a fluorescent dye. The capture antibody was 2B7 (directed against the 17 N-terminal amino acids of the HTT protein), and as detection antibody MW1 was used (binds to the expanded poly-glutamine domain of mutant HTT protein). The assay was performed as previously described (Fodale et al., J Huntingtons Dis. 2017; 6(4):349-361).

Results

Vector Genome Copies

Levels of vector DNA determined in brain punches at 6 and 12 months post-AAV5-miHTT injection are depicted in FIG. 17 (A). The highest levels (between 10⁶-10⁷ genome copies/ug DNA) were found in target areas (caudate and putamen), but also in other areas with direct neuronal connections to the target area, such as amygdala and thalamus. In cortical regions, vector DNA levels ranged between 10⁴ to 10⁵ genome copies/ug DNA. In more caudal regions, such as pons and cerebellum, and white matter regions, vector genome copies were the lowest (range 5×10³-10⁴ genome copies/ug DNA). In general, levels observed at 6 and 12 months post-administration were comparable.

miHTT Expression

A very widespread expression of miHTT was observed at 6 and 12 months post-injection (FIG. 18 A). Especially at 12 months, where more brain samples were assessed, all samples returned miHTT levels above the lower limit of quantification (except for one animal in the cerebellum). The expression of miHTT showed a good correlation with vector DNA levels (Pearson r 0.8963, p<0.001) (FIG. 18 B).

Mutant HTT Protein Lowering

The levels of mutant HTT protein were assessed both at 6 and 12 months, in naïve (control) and treated animals, and expressed as % from naïve control. Remarkable lowering was observed in most brain regions analyzed. Interestingly, the HTT % lowering was generally stronger at 12 than at 6 months for the non-target areas (that is, all brain regions except caudate and putamen) (FIG. 17 B). At 12 months post-injection, when an extensive bioanalysis was performed, mHTT protein lowering in treated animals with respect to controls was significant in all brain regions studied, except one (cerebellum) (FIG. 19). The widespread mutant HTT lowering across the entire minipig brain was beyond expectations based solely on distribution of the viral vector after local administration.

Global Correlation Analyses

To study the relationship between mutant HTT protein lowering and presence of vector DNA or miHTT in different brain regions of the minipig brain, correlation analyses were performed with all available data described above. A significant correlation between mutant HTT protein expression and vector DNA levels was obtained when adding all brain regions to the data analysis (Pearson r −0.3260, P<0.0001) (FIG. 20). From this graphical display, different observations could be made. First, the highest vector DNA levels corresponded to the strongest lowering, observed in the target regions (caudate and putamen), with vector DNA levels above 10⁶ gc/ug DNA on average and mutant HTT expression from control being less than 50%. Second, areas with direct connections to the target regions, such as cortical regions, thalamus and limbic regions, showed intermediate to strong mutant HTT protein lowering (ranging between 25 to 75% lowering), with vector DNA levels being above 10⁴ gc/ug DNA. Finally, areas with indirect connections to target regions, such as brainstem, hippocampus and cerebellum, as well as white matter, showed in cases strong mutant HTT protein lowering, while the vector DNA levels were below the lowest level calculated to be needed for therapeutic efficacy (that is, 10⁴ gc/ug DNA).

Regional Correlation Analyses

To study the relationship between vector DNA levels and mutant HTT protein lowering in target and non-target areas, we performed additional correlation analyses, separately for (i) target regions (caudate and putamen) (Figure X21A), (ii) regions with significant direct connections to target regions (thalamic nuclei, amygdala, nucleus accumbens and cortical regions) (Figure X21B) and (iii) regions with indirect connections to target regions (brainstem, hippocampus, cerebellum and white matter regions) (FIG. 21C). Negative correlations between vector DNA and mutant HTT protein expression were found for target and directly connected regions, the strongest correlation being for target regions (Pearson r −0.72, p<0.0001), than for directly connected regions (Pearson r −0.2758, p<0.0001); correlations between vector DNA and mutant HTT protein expression in indirectly connected regions did not reach significance (Pearson r −0.1871, p=0.108). Because not all cortical regions have significant direct connections with target regions (caudate and putamen), a separate analysis was performed for cortical subregions with direct and with indirect connections to caudate and/or putamen (FIG. 22). In line with the observations for the other brain regions with direct/indirect connections, in cortical regions directly connected to target regions a significant correlation was observed between vector DNA and mutant HTT protein levels (Pearson r −0.3734, p<0.0007), while no significant correlation was seen in cortical regions with indirect connections to caudate and/or putamen (Pearson r −0.1157, p=0.2831).

Conclusions

This example shows long-term (6 to 12 months post-injection) efficacy of AAV5-miHTT treatment in tgHD minipigs, bilaterally injected into caudate and putamen using real-time MRI-guided CED. Robust vector DNA levels, miHTT expression and mutant HTT protein was observed in target regions (caudate, putamen) and non-target regions with direct (thalamus, nucleus accumbens, subcortical regions) or indirect (brainstem, hippocampus, cerebellum, white matter) connections to caudate and/or putamen. Strikingly, regions with indirect connections to target areas showed significant mutant HTT protein lowering, while vector DNA levels in these regions were below the minimum threshold needed for efficacy. Correlation analyses strongly suggest that in regions with no direct connections with target regions, vector DNA levels alone do not correlate with the level of mutant HTT protein lowering. This supports the view of EV-mediated miHTT transfer to these regions being an important contributor to the widespread regional efficacy of AAV5-miHTT gene therapy, especially long-term and in areas where the vector cannot efficiently reach based on neuronal connections.

### Example 5

miHTT is Associated in Extravascular and Lipoprotein Fractions from NHP CSF

In this study, the association of miHTT to extracellular vesicle and/or lipoprotein fractions obtained from cerebrospinal fluid (CSF) from non-human primates (NHPs) injected with AAV5-miHTT in caudate and putamen was determined. The miHTT association profile was compared to the profile of endogenous miRNAs known to be expressed in CSF.

Samples

CSF samples from a study in NHP were used. In the study, adult cynomolgus monkeys were injected with AAV5-miHTT locally in caudate and putamen (dose 1-E13 gc/brain). Several longitudinal CSF samples were taken, namely on day 1 (pre-dose & post-dose 1 hr), week 2, week 4, week 8 and week 12 (prior to termination). For CSF sample collection, animals were sedated with Ketamine (10 mg/kg) and dexmedetomidine (0.015 mg/kg). Each animal's neck was shaved and its skin prepared for CSF collection. The neck was flexed and a 23 gauge needle was manually inserted in between the skull base and Cl into the cisterna magna gradually until CSF flow was established. CSF was collected upon verification of CSF flow into the needle hub and then 1.0 mL CSF sample was collected at each time point, centrifuged for 15 min at 2400 RPM, 4° C. and then divided into 2 aliquots, frozen on dry ice and stored at −70° C. until further analysis.

Methods

Size Exclusion Chomatography (Sec)

Only samples from pooled CSF of treated animals at week 2 post-injection were used for this analysis. Samples from two animals, 500 uL each sample, were thawed in a 37° C. bath and mixed in a single Eppendorf, resulting in a final volume of 1 mL. The mix was added to a size exclusion chromatography column (custom-made) and eluted with PBS, as previously described (Boing et al., (2014) Journal of Extracellular Vesicles, 3(1), 1-11). A total of 26 fractions of 500 uL each were collected and stored at −80° C. until further use.

RNA Isolation and RT-QPCR

A volume of 200 uL of each of the obtained SEC fractions (1-26) was used for RNA isolation using the Serum/Plasma Advanced Kit (QIAgen) and following the manufacturer's instructions. The RNA was eluted in a 16 uL of RNAse-free water. The microRNAs of interest (miHTT, miR16 and miR21) were quantified using specific Taqman assays designed for this purpose. Total RNA was used for cDNA synthesis, using TaqMan MicroRNA Reverse Transcription Kit (Applied Biosystems) together with gene-specific RT primers targeting the miRNAs of interest. For miHTT, a single stranded miHTT RNA standard line was taken along. Next, gene-specific TaqMan qPCR was performed with miHTT, miR16 or miR21-specific probes using TaqMan Fast Universal PCR Master Mix (Applied Biosystems). Using the miHTT standard line, miHTT molecules per reaction were determined, and expressed per uL of CSF used. For miR16 and miR21, the relative expression of one sample with respect to the other analyzed samples was calculated (assuming an amplification efficiency of 2).

Results

All the analyzed miRNAs, miHTT, miR16 and miR21, were successfully detected in SEC fractions obtained from NHP CSF (two weeks post AAV5-miHTT injection) (FIG. 23). Interestingly, miHTT was associated to both vesicle (fractions 7-10) and protein (fractions 17-21) fractions. In contrast, the endogenous miRNAs miR-16 and miR-21 were primarily associated to the protein fractions (fractions 17-21).

Conclusions

Using SEC, one of most widely accepted approaches to separate extracellular vesicles from biofluids, it was shown that miHTT is successfully detected, in both extracellular vesicle and lipoprotein fractions from NHP CSF 2 weeks after intraparenchymal injection of AAV5-miHTT. This observation supports the potent bystander effect of local miHTT treatment, i.e. miHTT being spread between brain regions through extracellular vesicles.

### FIGURES

FIGS. 1A-1B. FIG. 1A. Schematic of miR451 scaffold RNA structure indicating the first RNA sequence as it is designed. FIG. 1A discloses SEQ ID NO: 27. FIG. 1B. Schematic of expression cassette of a miRNA scaffold.

FIG. 2. DNA sequence of an expression construct (SEQ ID NO. 1) encoding a miR451 scaffold comprising a first RNA sequence of 22 nucleotides targeting a sequence of human ATXN3. The expression cassette comprises a CAG promotor shown in bold (position 43-1712), the sequence encoding the first RNA sequence that replaces the miRNA is shown in bold and underlined (position 2031-2052), followed by a second RNA sequence shown underlined (position 2053-2070), the hGH poly A signal shown in bold and italics (2318-2414). The first RNA sequence corresponds with the sequence that targets ATXN3, i.e. SEQ ID NO. 2. The pri-miRNA sequence comprises a pre-miRNA sequence. The pri-miRNA encoding sequence is shown between [brackets] (position 2015-2086).

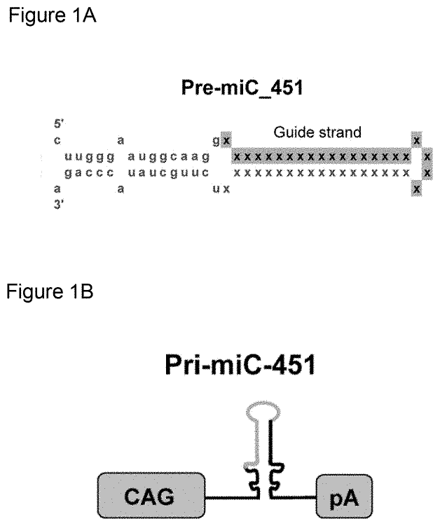

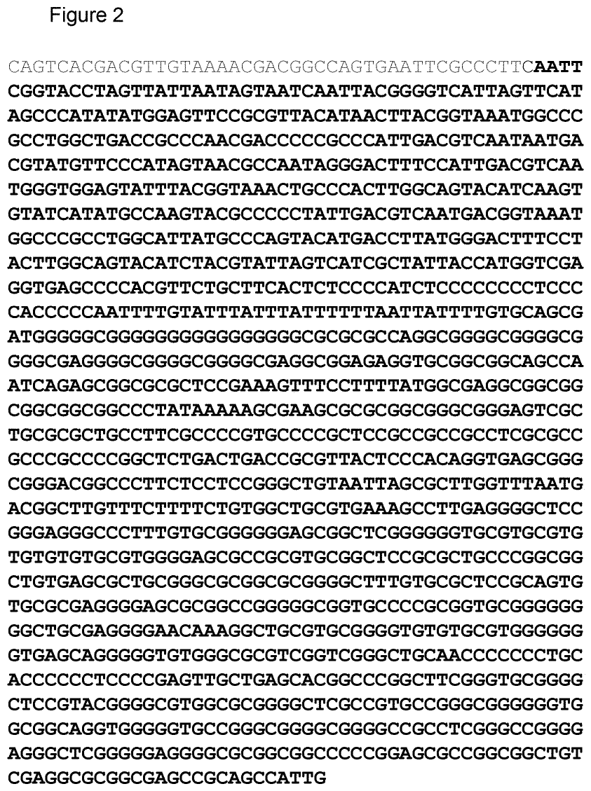

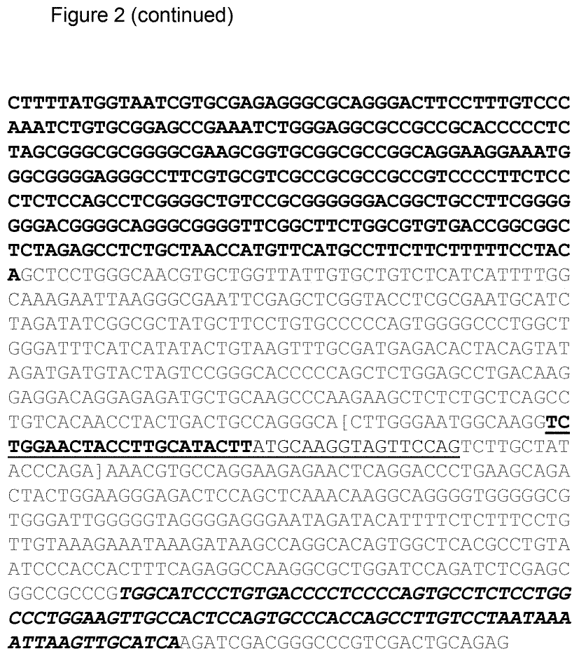

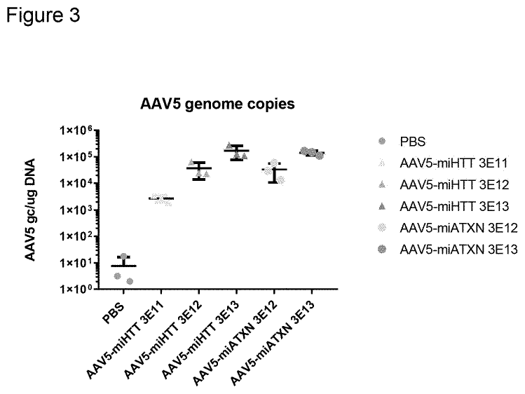

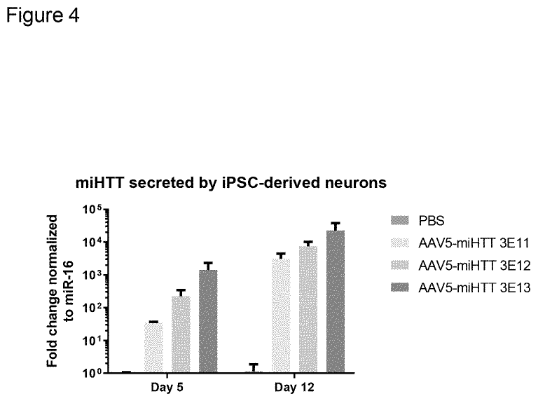

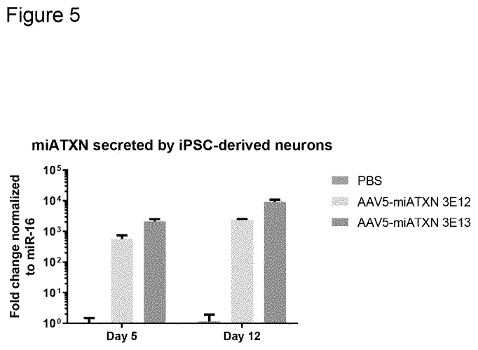

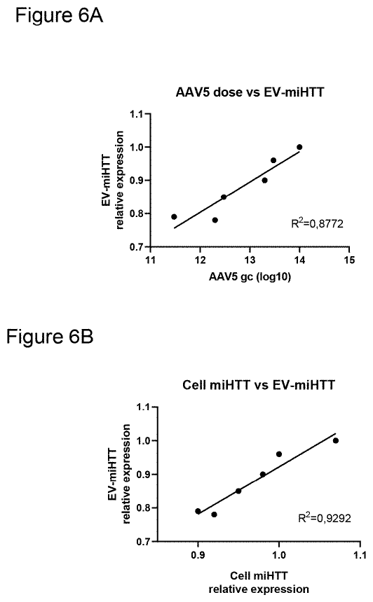

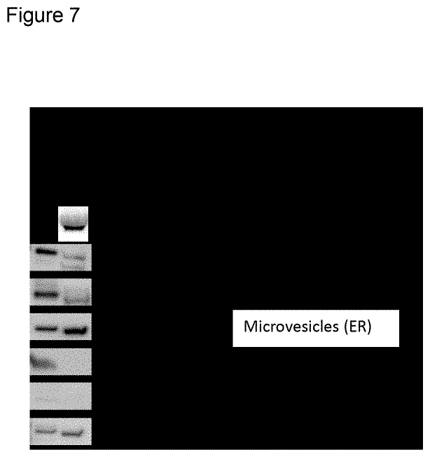

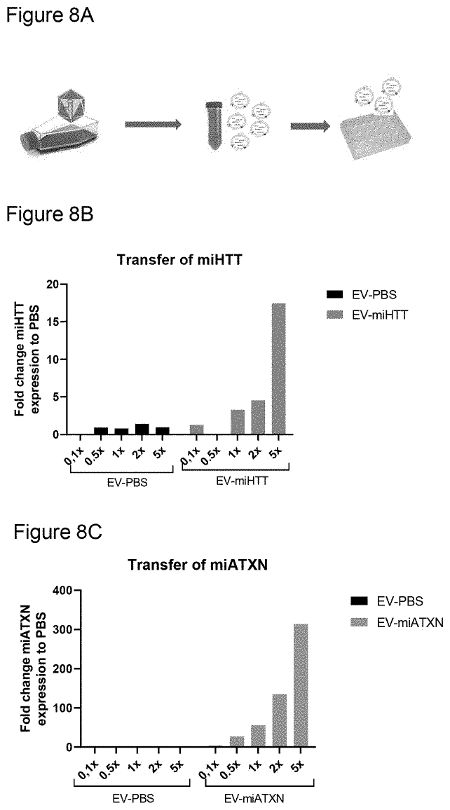

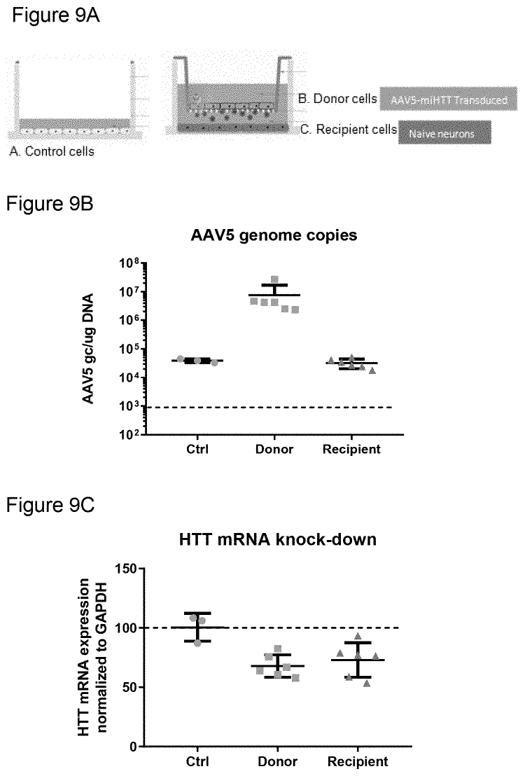

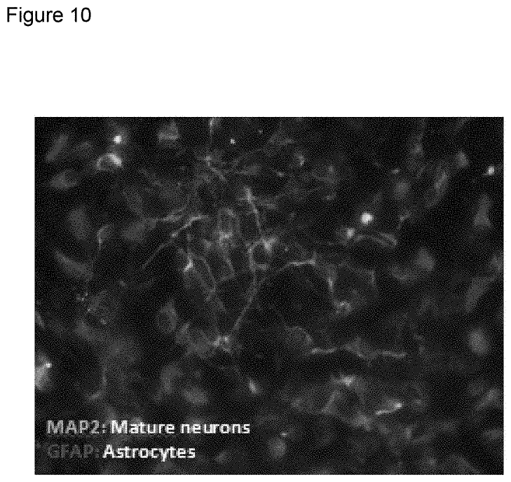

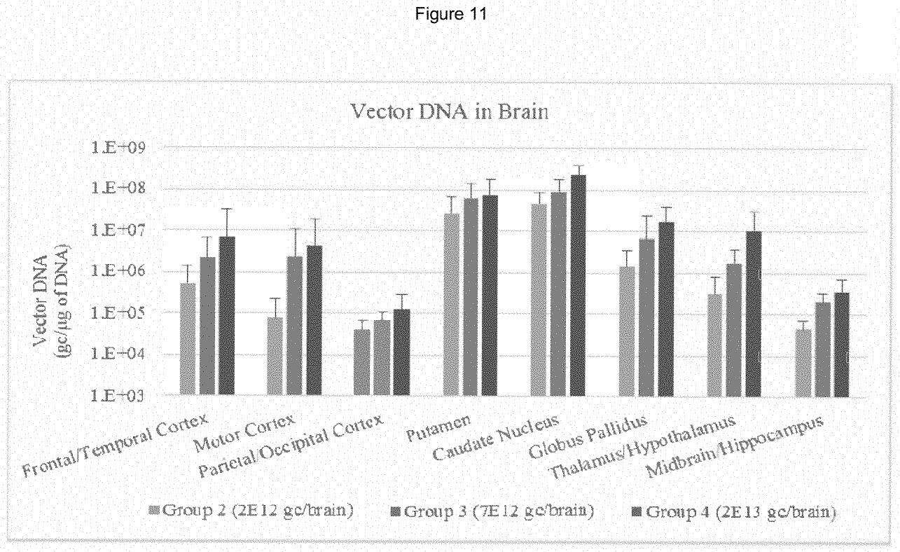

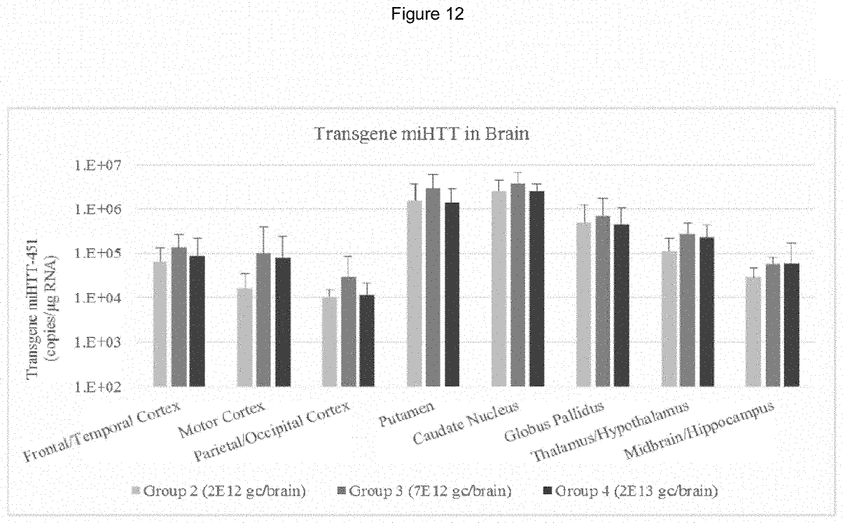

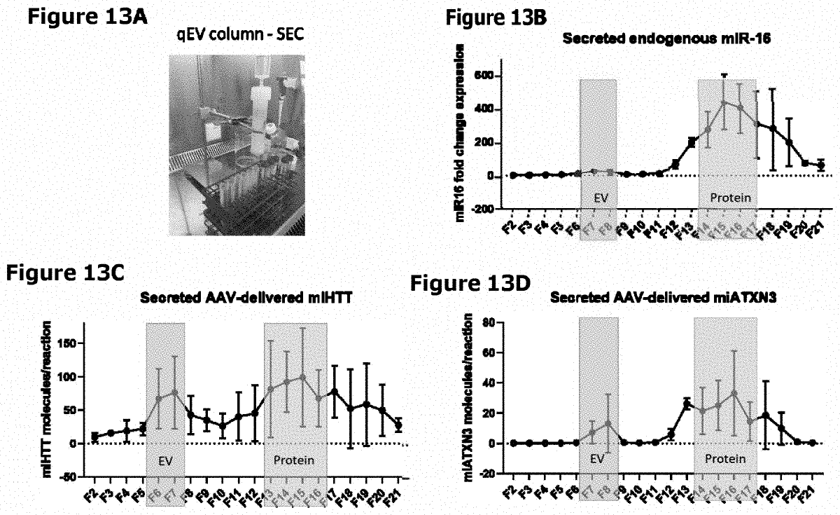

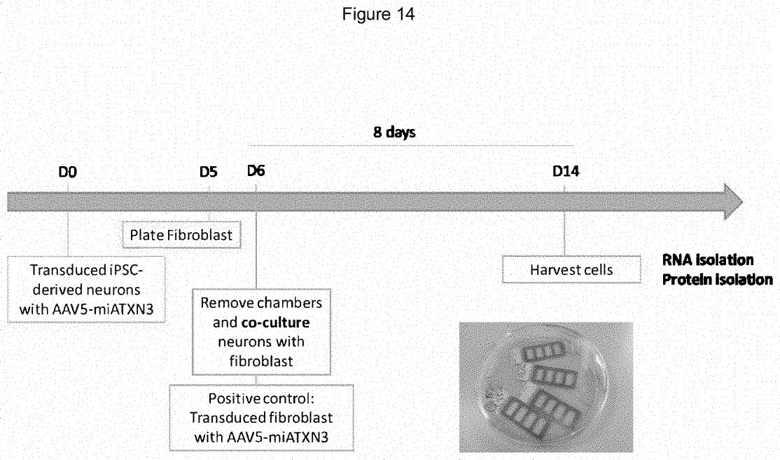

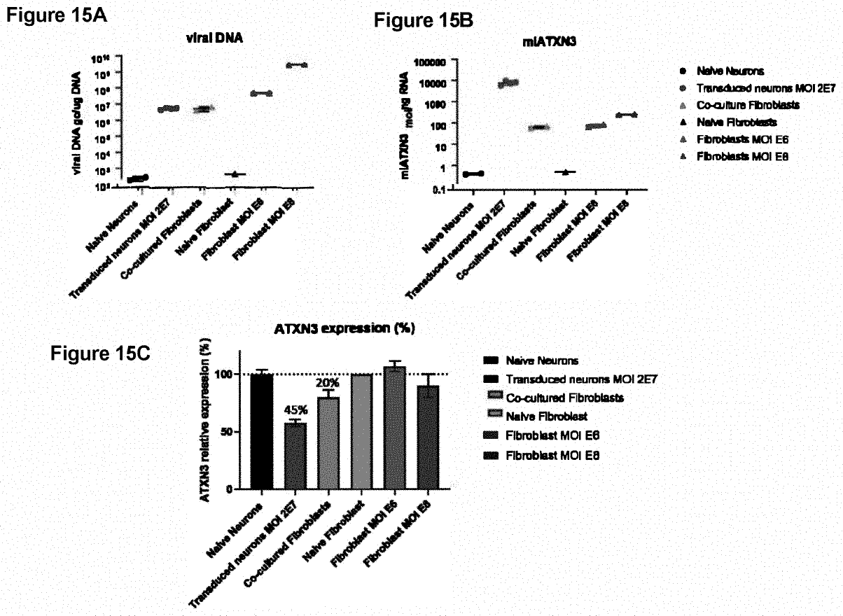

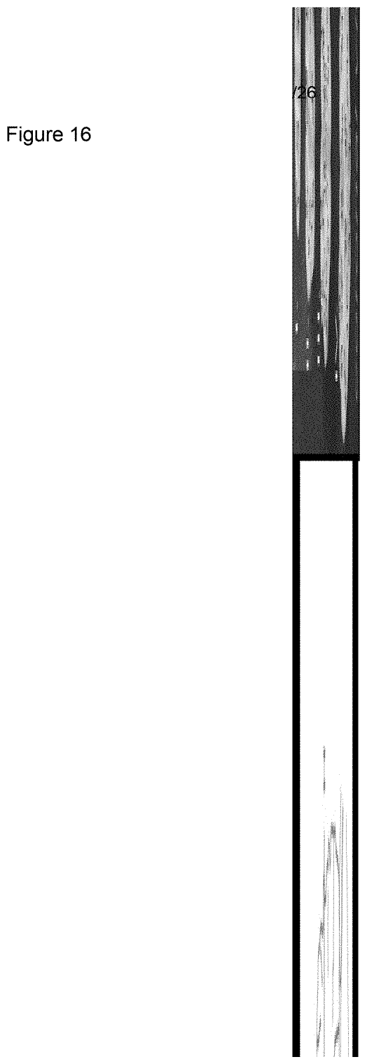

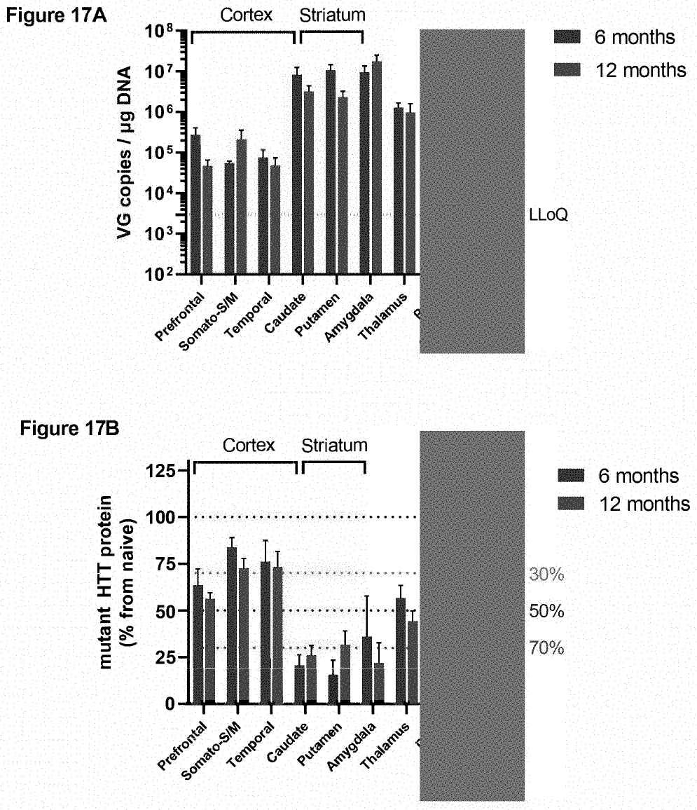

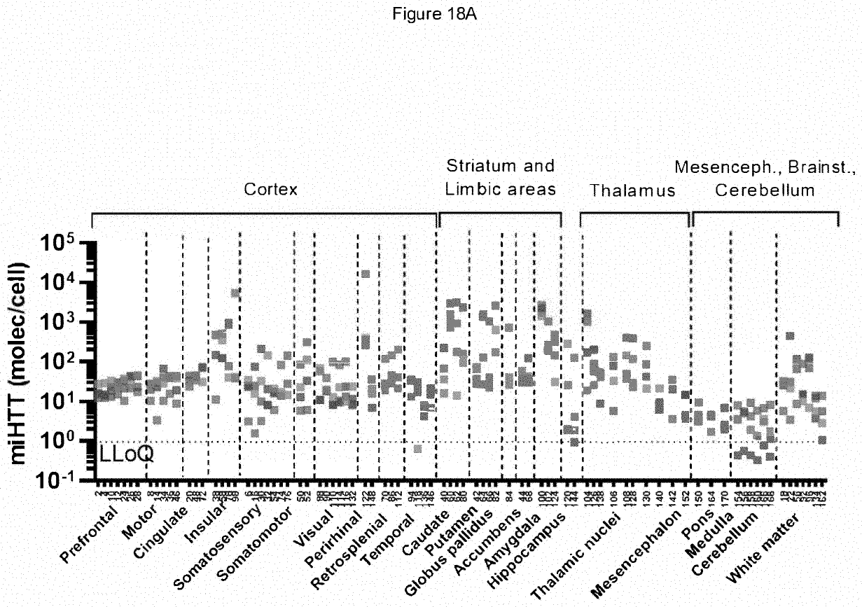

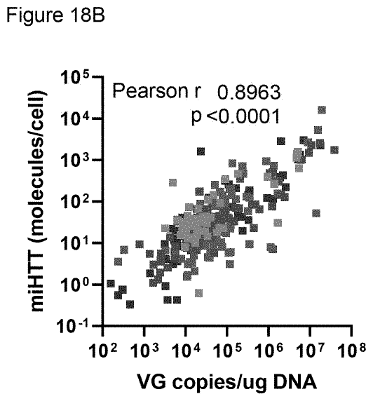

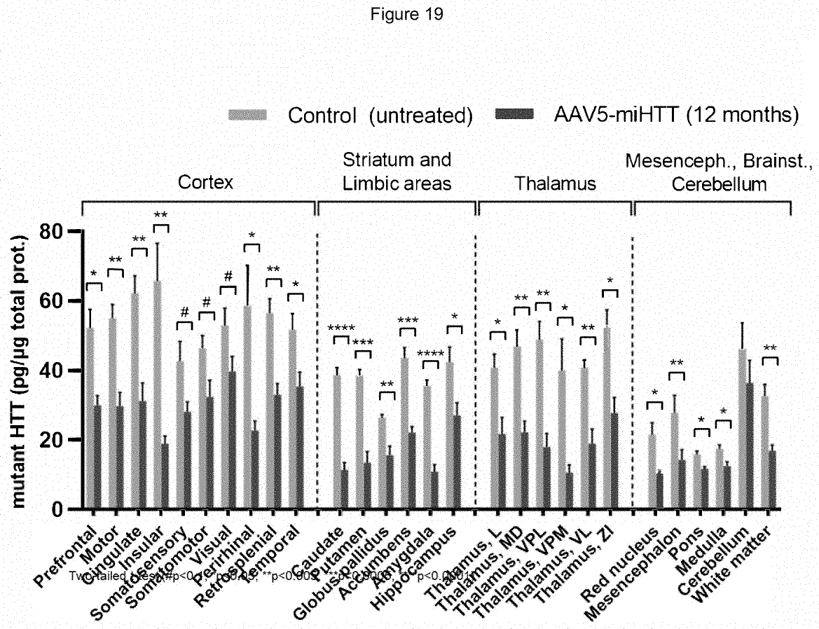

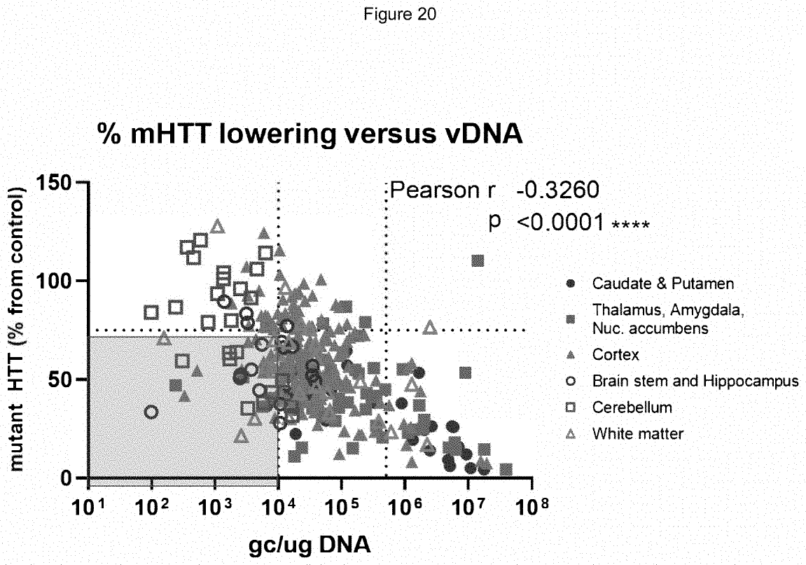

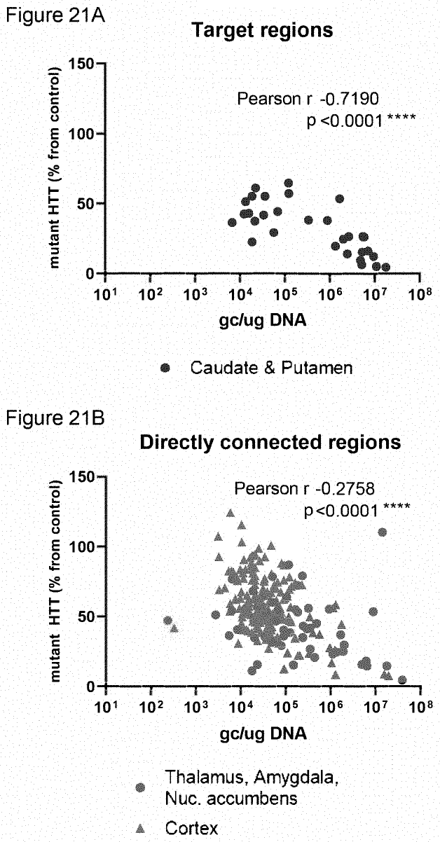

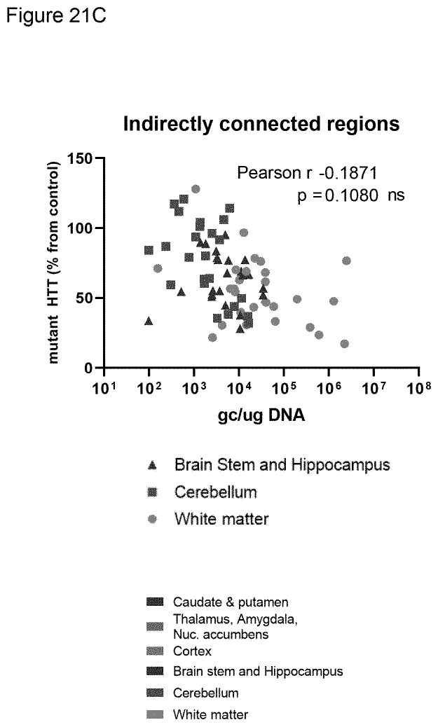

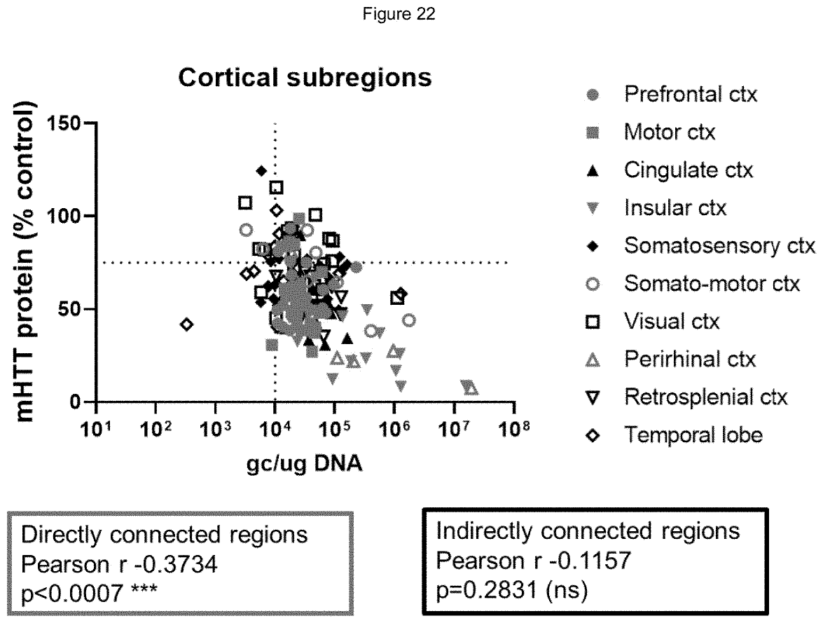

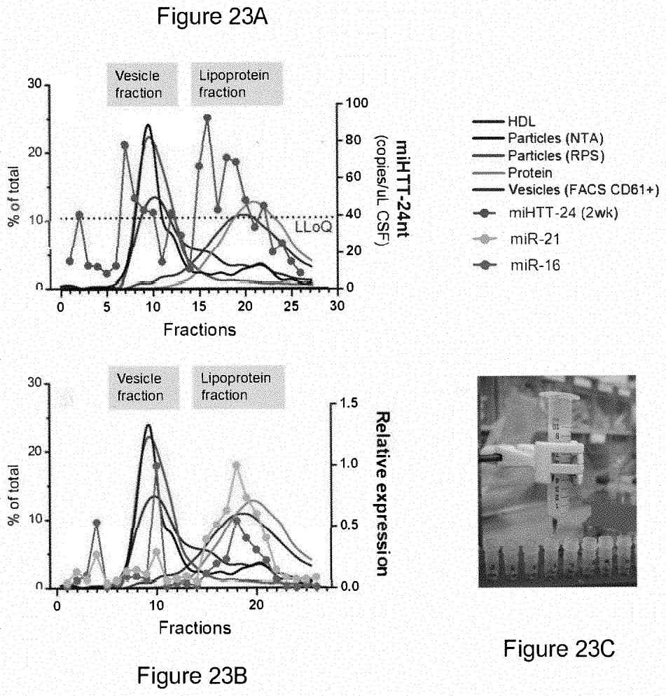

The sequence corresponding with the sequence encoding a miRNA designed to target the Huntington gene is 5′-CTTGGGAATGGCAAGGAAGGACTTGAGGGACTCGAAGACGAGTCCCTCAAGTCCTCTCT TGCTATACCCAGA-3′ (SEQ ID NO. 4) which sequence can replace the sequence in between [brackets], thereby obtaining an expression cassette for the miRNA targeting Huntington. The pre-miRNA sequence comprises the first RNA sequence and the second RNA sequence and the sequence encoding it is shown underlined, either normal or bold, (position 2031-2070). The pre-miRNA or pri-miRNA encoding sequence may be replaced e.g. by another sequence encoding a pre-miRNA. The first RNA sequence of the pre-miRNA or pri-miRNA can be any sequence of 22 nucleotides selected to bind and target a sequence in e.g. the ATXN3 gene or the HTT gene, or any other suitable target sequence. The second RNA sequence is selected and adapted to be complementary to the first RNA sequence. The secondary structure is checked on mfold by folding the RNA sequence using standard settings utilizing the RNA folding form, with folding temperature fixed at 37 degrees Celcius (as available online <URL:http://unafold.rna.albany.edu/?q=mfold>; Zuker et al., Nucleic Acids Res. 31 (13), 3406-15, (2003)) for folding, and adapted if necessary, into a miR-451 pri-miRNA structure as depicted in FIG. 1.

FIG. 3. Dose-dependent transduction of HD-iPSC derived neuronal cells with three doses of AAV5-miHTT (3E11gc, 3E12gc and 3E13gc) and two doses of AAV5-miATXN (3E12gc and 3E13gc) at 20 days after transduction. Results are expressed as AAV5 gc/ug genomic DNA (average+/−SEM of 3 wells per condition).

FIG. 4. Dose-dependent expression of miHTT in extracellular vesicles (EVs) isolated from medium of control and AAV5-miHTT transduced HD-iPSC derived neuronal cells. Results are expressed as miHTT levels with respect to an endogenous miRNA (miR-16), and with respect to levels in medium of control (PBS-treated) cells (average+/−SEM).

FIG. 5. Dose-dependent expression of miATXN in extracellular vesicles (EVs) isolated from medium of control and AAV5-miATXN transduced HD-iPSC derived neuronal cells. Results are expressed as miATXN levels with respect to an endogenous miRNA (miR-16), and with respect to levels in medium of control (PBS-treated) cells (average+/−SEM).

FIGS. 6A-6B. Summary data from n=6 independent experiments showing a robust correlation between secreted miHTT molecules detected in the medium of transduced cells and both FIG. 6A viral dose (expressed as log 10 of AAV5 genome copies) and FIG. 6B miHTT expression within the cells (expressed as miHTT relative expression).

FIG. 7. Composition of EVs precipitated from the medium, analyzed with different markers by western blot. Both total cell lysate and EV fraction after Exoquick precipitation are shown. In the EV precipitate we detected EV and exosomal markers (CD63, Alix and TSG-101), microvesicle markers (Calnexin) and proteins from RISC complex (Ago2) to which functional miRNAs are bound. Cellular markers (a-tubulin and ATPase) were used as controls to confirm the absence of cells or cellular debris.

FIGS. 8A-8C. FIG. 8A dose-dependent transfer of FIG. 8B miHTT and FIG. 8C miATXN to naïve neuronal cells. Medium from PBS, AAV5-miHTT and AAV5-miATXN transduced iPSC-derived neuronal cells, and EV isolated from the medium FIG. 8A. The EVs derived from the medium were added in different concentrations (0.1×, 0.5× 1×, 2× or 5×) to naïve iPSC-derived neuronal cells. Cells were harvested 24 hours after EV-transfer, and levels of miHTT or miATXN were measured (expressed as fold change with respect to PBS group).

FIGS. 9A-9C. Functional transfer of therapeutic miHTT between cells. FIG. 9A Experimental setup: transwell experiment in which iPS-derived cells (transduced with AAV5-miHTT) were seeded in polyester membrane cell culture inserts, and placed in a 6-well plate with naïve iPS-derived neurons. FIG. 9B AAV5 genome copies in control, donor and recipient cells; only donor cells had detectable AAV5 genome copies. FIG. 9C Knock-down of huntingtin mRNA (normalized to GADPH and expressed as % from control) in both donor and recipient cells (on average 30% knock-down with respect to control cells).

FIG. 10. Representative culture of iPSC-derived neuronal cells, immunocytochemically stained with markers of mature neurons (MAP2) and astrocytes (GFAP).

FIG. 11. Vector DNA levels (expressed in genome copies per ug of genomic DNA) in different brain regions of brain cynomolgous monkeys, injected with increasing doses of AAV5-miHTT (groups 2 to 4 as indicated in legend) (n=6 animals/group) by MRI-guided CED in putamen and caudate. Bars represent average+/−SD.

FIG. 12. miHTT levels (expressed in copies per ug of total RNA) in different brain regions of brain cynomolgous monkeys, injected with increasing doses of AAV5-miHTT (groups 2 to 4 as indicated in legend) (n=6 animals/group) by MRI-guided CED in putamen and caudate. Bars represent average+/−SD.

FIGS. 13A-13D: Detection of neuronal-secreted therapeutic miRNAs enriched in both EVs and protein-containing fractions by SEC. FIG. 13A) Image of qEV10 SEC column by Izon and collection of the different fractions. FIG. 13B) Fold change quantification of endogenous miR-16 in medium from AAV-transduced neuronal cells. miR-16 was found in association with proteins but not with EVs. FIGS. 13C and 13D) Quantification of abundance of therapeutic miRNAs (miHTT and miATXN3 respectively) in the medium of AAV-transduced neuronal cells. Both AAV-delivered miRNAs were enriched in both EVs and protein fractions.

FIG. 14: Experimental outline to investigate the functional transfer of therapeutic miRNAs in vitro. Neuronal cells, selected as secreting cells, were transduced with AAV-miATXN3 and co-culture together with fibroblast cells, selected as recipient cells for 8 days. (see methods)

FIGS. 15A-15C: Continuous transfer of secreted therapeutic miRNAs results in lowering of gene expression in recipient cells. FIG. 15A) Quantification of viral DNA genome copies shows an efficient transduction of neuronal cells, viral contamination of co-cultured fibroblast and high levels of viral DNA gc in directly transduced fibroblast. FIG. 15B) Quantification of miATXN3 molecules shows a high expression of miATXN3 molecules in neuronal cells and similar level of miATXN3 molecules in both co-cultured fibroblast and directly-transduced fibroblast. FIG. 15C) Relative expression of ATXN3 mRNA normalized to naïve cells of each group. There is a 45% lowering in AV-transduced neuronal cells, 20% lowering in co-cultured fibroblast and subtle or no lowering in directly transduced fibroblast.

FIG. 16. Dissection scheme of tgHD minipig brains. Brains were collected and sliced coronally (4 mm-thick sections), in regular intervals as indicated in the illustration on the right, collecting a total of 12 sections (indicated in roman numbers from I to XII). Thereafter brain punches of 3 mm in diameter were taken bilaterally. In animals sacrificed after 6 months, 54 punches were taken (red circles). In animals sacrificed after 12 months, a total of 170 punches were taken (red plus black circles). Each punch of the left hemisphere (even numbers) was divided in four parts for different purposes (DNA, RNA, protein or backup), while the punches from the right hemisphere (odd numbers) were not divided and kept as a backup.

FIGS. 17A-17B. FIG. 17A) Vector genome (VG) copies per μg of genomic DNA and FIG. 17B) mutant HTT protein (as % from naïve controls) in different brain regions of tgHD minipigs, at 6 (left bars) and 12 (right bars) months after intraparenchymal (caudate+putamen) MRI-guided CED administration of AAV5-miHTT. The shaded region indicates areas with relative low VG levels where a stronger mutant HTT protein lowering is observed at 12 months. LLoQ: lower limit of quantification.

FIGS. 18A-18B. FIG. 18A) Expression of miHTT (molecules/cell) in different brain regions of tgHD minipigs at 12 months after intraparenchymal (caudate+putamen) MRI-guided CED administration of AAV5-miHTT. Specific brain regions are indicated in the x-axes, together with the numbering of the dissection punches. Each square indicates the levels of a single punch per animal in any given brain region. LLoQ: lower limit of quantification. FIG. 18B) Correlation of miHTT (molecules/cell) with vector genome (VG) copies per μg of genomic DNA in different brain regions of tgHD minipigs at 12 months post-administration. A significant positive correlation was obtained (Pearson r 0.8963, p<0.0001).

FIG. 19. Mutant HTT protein levels (pg/μg total protein) in different brain regions of tgHD minipigs. Animals were sacrificed under control (untreated) conditions (left bars) or at 12 months (right bars) after intraparenchymal (caudate+putamen) MRI-guided CED administration of AAV5-miHTT. Significant differences between control and treated groups are indicated as *<0.05, **<0.005, ***0.0005, ****0.0001 or #0.1 (Student's t=test, corrected p value).

FIG. 20. Correlation of mutant HTT protein (as % from naïve controls) and vector genome (VG) copies per μg of genomic DNA in different brain regions of tgHD minipigs at 12 months post-administration of AAV5-miHTT in caudate and putamen. A significant negative correlation was obtained (Pearson r −0.3260, p<0.0001). Lines crossing the x-axis indicate the threshold estimated to be needed for efficacy of HTT lowering (10⁴ VG/μg DNA) and the minimum levels found in target areas (8×10⁵ VG/μg DNA). Line crossing the y-axis delimitates the efficacy threshold (75% mutant HTT expression with respect to control). The shadowed region delimitates punches where VG levels below the efficacy threshold showed mutant HTT expression below 75% from control.

FIGS. 21A-21C. Mutant HTT protein levels (as % from control) in FIG. 21A) target regions (caudate and putamen), FIG. 21B) regions directly connected to target regions (thalamus, amygdala, nucleus accumbens and cortex) and FIG. 21C) regions with indirect connections to target regions (brainstem, hippocampus, cerebellum and white matter). Pearson correlations with vector genome (VG)/μg DNA levels led to significant negative correlations in target regions (r −0.7190) and directly connected regions (r −0.2758), but not in regions with indirect connections (r −0.1871, p=0.1080).

FIG. 22. Mutant HTT protein levels (as % from control) in cortical regions directly or indirectly connected to target regions (caudate and putamen) of tgHD minipigs at 12 months post-administration of AAV5-miHTT. Cortical regions with direct connections include prefrontal, motor, insular somato-motor and perirhinal cortices. Cortical regions with indirect connections comprise cingulate, somatosensory, visual, retrosplenial and temporal cortices. Pearson correlations with vector genome (VG)/μg DNA levels led to significant negative correlations in directly connected regions (r −0.3734, p<0.0007) but not in regions with indirect connections (r −0.1157, p=0.2831).

FIGS. 23A-23C. FIG. 23A) Relative expression of miHTT in cerebrospinal fluid (CSF) of non-human primates (NHPs), two weeks after intrastriatal administration of AAV5-miHTT. Using size exclusion chromatography (SEC), miHTT was determined in both vesicle and (lipo)protein fractions. FIG. 23B) Relative expression of two endogenous microRNAs, miR-21 and miR-16, in SEC fractions from NHP CSF. FIG. 23C) Representative SEC column used to separate vesicle and (lipo)protein fractions from NHP CSF.

1. A gene delivery vehicle comprising a miRNA scaffold for delivery of a miRNA to a target cell resulting in silencing of a desired gene in the transduced target cell, whereby spread of the miRNA to other non-transduced target cells results in silencing of the desired gene in the non transduced target cells.

2. The gene delivery vehicle according to claim 1, wherein the gene delivery vehicle comprises a mir-451 scaffold or a functional equivalent thereof.

3. The gene delivery vehicle according to claim 1, whereby the spread occurs through packaging of the miRNA in extracellular vesicles.

4. The gene delivery vehicle according to claim 3, wherein the extracellular vesicles are exosomes or microvesicles.

5. The gene delivery vehicle according to claim 1, wherein the silencing comprises nuclear silencing.

6. The gene delivery vehicle according to claim 1, wherein the silencing comprises cytoplasmic silencing.

7. The gene delivery vehicle according to claim 1, wherein the gene delivery vehicle is a virus derived particle.

8. The gene delivery vehicle according to claim 7, wherein the virus derived particle is an AAV based particle.

9. The gene delivery vehicle according to claim 1, wherein the miRNA is under control of a relatively weak promoter.

10. The gene delivery vehicle according to claim 9, wherein the promoter is selected from the group consisting of Polymerase II promotor, a chicken-beta actin promoter, a CAG promoter, an EF1alpha promoter, a PGK promoter and a tissue-specific promoter.

11. A method of treating a neurodegenerative disease, comprising administering to a subject in need thereof a gene delivery vehicle according to claim 1.

12. The method according to claim 9, wherein the neurodegenerative disease is selected from the group consisting of Huntington's disease, amyotrophic lateral sclerosis (ALS), spinocerebellar ataxias, Parkinson's disease, Alzheimer's disease, and frontotemporal dementia (FTD).

13. The method according to claim 9, wherein the miRNA is delivered to a brain cell resulting in silencing of a desired gene and spread of the miRNA to other brain cells for silencing of the desired gene, wherein the gene delivery vehicle comprises a mir-451 scaffold or a functional equivalent thereof.

14. The method according to claim 9, wherein the administration is intravenous, intrathecal, intraparenchymal, intravitreal, or subretinal administration.

15. The method according to claim 14, wherein the administration is intraportal or intracoronary delivery or isolated limb perfusion.

16. The method according to claim 13, wherein the miRNA is delivered wherein the miRNA is under control of a relatively weak promoter, such as a promoter is selected from the group consisting of a Polymerase II promotor, a chicken-beta actin promoter, an EF1alpha promoter, a CAG promoter, a PGK promoter or a tissue-specific promoter for liver expression such as LP1, or AAT.

17. A method of treating a liver disease or metabolic disorder, comprising administering to a subject in need thereof a gene delivery vehicle according to claim 1.

18. The method according to claim 9, wherein the miRNA is delivered to a liver cell resulting in silencing of a desired gene and spread of the miRNA to other liver cells for silencing of the desired gene, wherein the gene delivery vehicle comprises a mir-451 scaffold or a functional equivalent thereof.

19. A pharmaceutical composition comprising a gene delivery vehicle according to claim 1 and suitable excipients.


SEQUENCE LISTING (27 sequences)

SEQ ID NO: 1 - DNA, 2440 bp, Artificial Sequence
  Description of Artificial Sequence Synthetic polynucleotide
  Expression construct
cagtcacgacgttgtaaaacgacggccagtgaattcgcccttcaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgcttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaagggcgaattcgagctcggtacctcgcgaatgcatctagatatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtctggaactaccttgcatacttatgcaaggtagttccagtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatccagatctcgagcggccgcccgtggcatccctgtgacccctccccagtgcctctcctggccctggaagttgccactccagtgcccaccagccttgtcctaataaaattaagttgcatcaagatcgacgggcccgtcgactgcagag

SEQ ID NO: 2 - DNA, 22 bp, Homo sapiens
  ATXN3
tctggaactaccttgcatactt

SEQ ID NO: 3 - DNA, 22 bp, Homo sapiens
  HTT
aaggacttgagggactcgaaga

SEQ ID NO: 4 - DNA, 72 bp, Artificial Sequence
  Description of Artificial Sequence Synthetic oligonucleotide
  HTT miRNA encoding sequence
cttgggaatggcaaggaaggacttgagggactcgaagacgagtccctcaagtcctctcttgctatacccaga

SEQ ID NO: 5 - RNA, 22 nt, Artificial Sequence
  Description of Artificial Sequence Synthetic oligonucleotide
  RNA sequences complementary to HTT
aaggacuugagggacucgaaga

SEQ ID NO: 6 - RNA, 22 nt, Artificial Sequence
  Description of Artificial Sequence Synthetic oligonucleotide
  RNA sequences complementary to ATNX3
ucuggaacuaccuugcauacuu

SEQ ID NO: 7 - DNA, 24 bp, Artificial Sequence
  Description of Artificial Sequence Synthetic oligonucleotide
  Small RNAseq analysis of sequences produced from the scaffold targeting Huntington
aaggacttgagggactcgaagacg

SEQ ID NO: 8 - DNA, 30 bp, Artificial Sequence
  Description of Artificial Sequence Synthetic oligonucleotide
  Small RNAseq analysis of sequences produced from the scaffold targeting Huntington
aaggacttgagggactcgaagacgagtccc

SEQ ID NO: 9 - DNA, 23 bp, Artificial Sequence
  Description of Artificial Sequence Synthetic oligonucleotide
  Small RNAseq analysis of sequences produced from the scaffold targeting Huntington
aaggacttgagggactcgaagac

SEQ ID NO: 10 - DNA, 25 bp, Artificial Sequence
  Description of Artificial Sequence Synthetic oligonucleotide
  Small RNAseq analysis of sequences produced from the scaffold targeting Huntington
aaggacttgagggactcgaagacga

SEQ ID NO: 11 - DNA, 21 bp, Artificial Sequence
  Description of Artificial Sequence Synthetic oligonucleotide
  Small RNAseq analysis of sequences produced from the scaffold targeting Huntington
aaggacttgagggactcgaag

SEQ ID NO: 12 - DNA, 27 bp, Artificial Sequence
  Description of Artificial Sequence Synthetic oligonucleotide
  Small RNAseq analysis of sequences produced from the scaffold targeting Huntington
aaggacttgagggactcgaagacgagt

SEQ ID NO: 13 - DNA, 19 bp, Artificial Sequence
  Description of Artificial Sequence Synthetic oligonucleotide
  Small RNAseq analysis of sequences produced from the scaffold targeting Huntington
aaggacttgagggactcga

SEQ ID NO: 14 - DNA, 31 bp, Artificial Sequence
  Description of Artificial Sequence Synthetic oligonucleotide
  Small RNAseq analysis of sequences produced from the scaffold targeting Huntington
aaggacttgagggactcgaagacgagtccct

SEQ ID NO: 15 - DNA, 20 bp, Artificial Sequence
  Description of Artificial Sequence Synthetic oligonucleotide
  Small RNAseq analysis of sequences produced from the scaffold targeting Huntington
aaggacttgagggactcgaa

SEQ ID NO: 16 - DNA, 24 bp, Artificial Sequence
  Description of Artificial Sequence Synthetic oligonucleotide
  Small RNAseq analysis of tsequences produced from the scaffold targeting ATXN3
tctggaactaccttgcatacttat

SEQ ID NO: 17 - DNA, 23 bp, Artificial Sequence
  Description of Artificial Sequence Synthetic oligonucleotide
  Small RNAseq analysis of tsequences produced from the scaffold targeting ATXN3
tctggaactaccttgcatactta

SEQ ID NO: 18 - DNA, 21 bp, Artificial Sequence
  Description of Artificial Sequence Synthetic oligonucleotide
  Small RNAseq analysis of tsequences produced from the scaffold targeting ATXN3
tctggaactaccttgcatact

SEQ ID NO: 19 - DNA, 30 bp, Artificial Sequence
  Description of Artificial Sequence Synthetic oligonucleotide
  Small RNAseq analysis of tsequences produced from the scaffold targeting ATXN3
tctggaactaccttgcatacttatgcaagg

SEQ ID NO: 20 - RNA, 16 nt, Artificial Sequence
  Description of Artificial Sequence Synthetic oligonucleotide
  miRNA451 scaffold 5′sequence
cuugggaauggcaagg

SEQ ID NO: 21 - RNA, 17 nt, Artificial Sequence
  Description of Artificial Sequence Synthetic oligonucleotide
  miRNA451 scaffold 3′ sequence
cucuugcuauacccaga

SEQ ID NO: 22 - DNA, 20 bp, Artificial Sequence
  Description of Artificial Sequence Synthetic oligonucleotide
  primer A (forward)
cccaccagccttgtcctaat

SEQ ID NO: 23 - DNA, 21 bp, Artificial Sequence
  Description of Artificial Sequence Synthetic oligonucleotide
  primer B (reverse)
gttcctcagatcagcttgcat

SEQ ID NO: 24 - DNA, 23 bp, Artificial Sequence
  Description of Artificial Sequence Synthetic oligonucleotide
  probe C (5′ 6FAM)
acgggcccgtcgactgcagaggc

SEQ ID NO: 25 - DNA, 16 bp, Artificial Sequence
  Description of Artificial Sequence Synthetic oligonucleotide
  forward primer
gagccgcagccattgc

SEQ ID NO: 26 - DNA, 23 bp, Artificial Sequence
  Description of Artificial Sequence Synthetic oligonucleotide
  reverse primer
cacagatttgggacaaaggaagt

SEQ ID NO: 27 - RNA, 72 nt, Artificial Sequence
  Description of Artificial Sequence Synthetic oligonucleotide
  Pre-miC_451
  modified_base (17)..(56): a, c, g or u
cuugggaauggcaaggnnnnnnnnnnnnnnnnnnnnnnnnnnnnnnnnnnnnnnnnucuugcuauacccaga